SEQUENCE LISTING (2 sequences)

SEQ ID NO: 1 - DNA, 21 bp, Artificial Sequence
  Chemically synthesized Artificial Sequence - RNA inhibitor of C-FLIP
aagcagtctgttcaaggagca

SEQ ID NO: 2 - DNA, 21 bp, Artificial Sequence
  Chemically synthesized Artificial Sequence - RNA inhibitor of C-FLIP
aaggaacagcttggcgctcaa

(19) United States
(12) Patent Application Publication
(10) Pub. No.: US 2008/0171051 A1
(43) Pub. Date: Jul. 17, 2008

(54) Cancer Treatment

(76) Inventors: Patrick Gerard Johnston, Belfast (GB); Daniel Longley, Lisburn (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 10/580,748

(22) PCT Filed: Nov. 26, 2004
(86) PCT No.: PCT/GB04/05006

§ 371 Date: Aug. 11, 2006

(30) Foreign Application Priority Data
Nov. 26, 2003 (GB) 0327493.3
Nov. 26, 2003 (GB) 0327499.0

(51) Int. Cl. A61K 39/395; A61K 48/00; A61K 31/7072; A61K 31/4745; A61K 31/282; G01N 33/574

(52) U.S. Cl. 424/155.1; 514/44; 514/49; 514/492; 514/283; 435/7.23

(57) ABSTRACT

The invention relates to methods and compositions for treatment of cancer. In one embodiment the method involves the use of a c-FLIP inhibitor as the sole active agent. In another embodiment the invention relates to the treatment of p53 mutant cancers using combinations of c-FLIP inhibitors and chemotherapeutic agents.

### FIELD OF THE INVENTION

The present invention relates to cancer treatment. In particular, it relates to methods and compositions for the treatment of cancer, including cancers characterised by p53 mutations.

### BACKGROUND TO THE INVENTION

5-FU4 is widely used in the treatment of a range of cancers including colorectal, breast and cancers of the aerodigestive tract. The mechanism of cytotoxicity of 5-FU has been ascribed to the misincorporation of fluoronucleotides into RNA and DNA and to the inhibition of the nucleotide synthetic enzyme thymidylate synthase (TS) (Longley et al., 2003). TS catalyses the conversion of deoxyuridine monophosphate (dUMP) to deoxythymidine monophosphate (dTMP) with 5,10-methylene tetrahydrofolate (CH2THF) as the methyl donor. This reaction provides the sole intracellular source of thymidylate, which is essential for DNA synthesis and repair. The 5-FU metabolite fluorodeoxyuridine monophosphate (FdUMP) forms a stable complex with TS and CH2THF resulting in enzyme inhibition (Longley et al., 2003). Recently, more specific folate-based inhibitors of TS have been developed such as tomudex (TDX) and Alimta (MTA), which form a stable complex with TS and dUMP that inhibits binding of CH₂THF to the enzyme (Hughes et al., 1999; Shih et al., 1997). TS inhibition causes nucleotide pool imbalances that result in S phase cell cycle arrest and apoptosis (Aherne et al., 1996; Longley et al., 2002; Longley et al., 2001). Oxaliplatin is a third generation platinum-based DNA damaging agent that is used in combination with 5-FU in the treatment of advanced colorectal cancer (Giacchetti et al., 2000). Drug resistance is a major factor limiting the effectiveness of chemotherapies. The topoisomerase-1 inhibitor irinotecan (CPT-11) and the DNA damaging agent oxaliplatin are now being used in conjunction with 5-FU for the treatment of metastatic colorectal cancer, having demonstrated improved response rates compared to treatment with 5-FU alone (40-50% compared to 10-15%) (10, 11). Despite these improvements, the vast majority of responding patients relapse, with median survival times of only 22-24 months. Clearly, new approaches are needed for the treatment of this disease.

Death receptors such as Fas and the TRAIL (tumour necrosis factor (TNF)-related apoptosis-inducing ligand) receptors DR4 (TRAIL-R1) and DR5 (TRAIL-R2) trigger death signals when bound by their natural ligands (1,2). Ligand binding to the death receptors leads to recruitment of the adaptor protein FADD (Fas-associated death domain), which in turn recruits procaspase 8 zymogens to from the death-inducing signalling complex (DISC) (Nagata, 1999). Procaspase 8 molecules become activated at the DISC and subsequently activate pro-apoptotic downstream molecules such as caspase 3 and BID. FasL expression is up-regulated in most colon tumours, and it has been postulated that tumour FasL induces apoptosis of Fas-sensitive immune effector cells (O'Connell et al., 1999). This mechanism of immune escape requires that tumour cells develop resistance to Fas-mediated apoptosis to prevent autocrine and paracrine tumour cell death.

A key inhibitor of Fas signaling is c-FLIP, which inhibits procaspase 8 recruitment and processing at the DISC (Krueger et al., 2001). Differential splicing gives rise to long (c-FLIP_{L}) and short (c-FLIP_{S}) forms of c-FLIP, both of which bind to FADD within the DISC. c-FLIP_{S} directly inhibits caspase 8 activation at the DISC, whereas c-FLIP_{L} is first cleaved to a p43 truncated form that inhibits complete processing of procaspase 8 to its active subunits. c-FLIP also inhibits procaspase 8 activation at DISCs formed by the TRAIL (TNF-related apoptosis-inducing ligand) death receptors DR4 (TRAIL-R1) and DR5 (TRAIL-R2) (Krueger et al., 2001). In addition to blocking caspase 8 activation, DISC-bound c-FLIP has been reported to promote activation of the ERK, PI3-kinase/Akt and NF-κB signaling pathways (Krueger et al., 2001). Thus, c-FLIP potentially converts death receptor signaling from pro- to anti-apoptotic by activating intrinsic survival pathways. Significantly, c-FLIP_{L} has been found to be overexpressed in colonic adenocarcinomas compared to matched normal tissue, suggesting that c-FLIP may contribute to in vivo tumour transformation (Ryu et al., 2001).

### SUMMARY OF THE INVENTION

As described herein and, as shown in our co-pending PCT application filed on the same day as the present application and claiming priority from GB patent application 0327493.3, the present inventors have shown that by combining treatment using a death receptor ligand, such as an anti FAS antibody, for example, CH-11, with a chemotherapeutic agent such as 5-FU or an antifolate drug, such as ralitrexed (RTX) or pemetrexed (MTA, Alimta), a synergistic effect is achieved in the killing of cancer cells. However, the synergistic effect achieved was abrogated in cancer cells which overexpress c-FLIP.

As described in the Examples, in cell lines which demonstrated overexpression of c-FLIP and associated resistance to chemotherapy e.g 5-FU induced apoptosis, inhibition of FLIP expression reversed the resistance to chemotherapy-induced apoptosis. On further investigating this effect, the inventors tested a number of cell lines having a p53 mutation or p53 null genotype.

To their surprise, the inventors observed that down-regulation of c-FLIP markedly enhanced apoptosis in response to certain chemotherapeutic agents in the p53 mutant cells, which are usually highly resistant to the particular chemotherapeutic agents. This surprising observation enables the use of combinations of such cFLIP inhibitors and chemotherapeutic agents in the treatment of cancers associated with p53 mutations.

Accordingly, in a first aspect of the present invention, there is provided a method of killing cancer cells having a p53 mutation, comprising administration to said cells of:

(a) a c-FLIP inhibitor and
(b) a chemotherapeutic agent, wherein the chemotherapeutic agent is a thymidylate synthase inhibitor, a platinum cytotoxic agent or a topoisomerase inhibitor.

In a second aspect, there is provided a method of treating cancer associated with a p53 mutation comprising administration to a subject in need thereof of

(a) a c-FLIP inhibitor and
(b) a chemotherapeutic agent, wherein the chemotherapeutic agent is a thymidylate synthase inhibitor, a platinum cytotoxic agent or a topoisomerase inhibitor.

A third aspect of the invention comprises the use of

(a) a c-FLIP inhibitor and
(b) a chemotherapeutic agent, wherein the chemotherapeutic agent is a thymidylate synthase inhibitor, a platinum cytotoxic agent or a topoisomerase inhibitor in the preparation of a medicament for treating cancer associated with a p53 mutation.

A fourth aspect provides a pharmaceutical composition for the treatment of a cancer associated with a p53 mutation, wherein the composition comprises (a) a c-FLIP inhibitor

(b) a chemotherapeutic agent, wherein the chemotherapeutic agent is a thymidylate synthase inhibitor, a platinum cytotoxic agent or a topoisomerase inhibitor and
(c) a pharmaceutically acceptable excipient, diluent or carrier.

A fifth aspect provides a kit for the treatment of cancer associated with a p53 mutation, said kit comprising

(a) a c-FLIP inhibitor and
(b) a chemotherapeutic agent, wherein the chemotherapeutic agent is a thymidylate synthase inhibitor, a platinum cytotoxic agent or a topoisomerase inhibitor and
(c) instructions for the administration of (a) and (b) separately, sequentially or simultaneously.

In any of the first to fifth aspects of the invention, the c-FLIP inhibitor and the chemotherapeutic agent may be provided and administered in the absence of other active agents. However, in a preferred embodiment of theses aspects aspects of the invention, there is provided (c) a death receptor binding member, or a nucleic acid encoding said binding member.

Any suitable death receptor binding member may be used. Death receptors include, Fas, TNFR, DR-3, DR-4 and DR-5. In preferred embodiments of the invention, the death receptor is FAS.

The c-FLIP inhibitor, the chemotherapeutic agent and where applicable the death receptor ligand, may be administered simultaneously, sequentially or simultaneously. In preferred embodiments of the invention, the c-FLIP inhibitor is administered prior to the chemotherapeutic agent and, where applicable, the specific binding member.

A preferred binding member for use in the invention is an antibody or a fragment thereof. In particularly preferred embodiments, the binding member is the FAS antibody CH11 (Yonehara, S., Ishii, A. and Yonehara, M. (1989) J. Exp. Med. 169, 1747-1756) (available commercially e.g. from Upstate Biotechnology, Lake Placid, N.Y.).

Any suitable thymidylate synthase inhibitor, platinum cytotoxic agent or topoisomerase inhibitor may be used in the present invention. Examples of thymidylate synthase inhibitors which may be used in the methods of the invention include 5-FU, MTA and TDX. In a preferred embodiment, the thymidylate synthase inhibitor is 5-FU. Examples of platinum cytotoxic agents which may be used include cisplatin and oxaliplatin. In a particularly preferred embodiment of the invention, the chemotherapeutic agent is cisplatin. Any suitable topoisomerase inhibitor may be used in the present invention. In a preferred embodiment, the topoisomerase inhibitor is a topoisomerase I inhibitor, for example a camptothecin. A suitable topoisomerase I, inhibitor, which may be used in the present invention is irenotecan (CPT-11). Unless, the context demand otherwise, reference to CPT-11 should be taken to encompass CPT-31 or its active metabolite SN-38.

In preferred embodiments of the invention, the c-FLIP inhibitor and the chemotherapeutic agent are administered in a potentiating ratio. the term “potentiating ratio” in the context of the present invention is used to indicate that the cFLIP inhibitor and chemotherapeutic agent are present in a ratio such that the cytotoxic activity of the combination is greater than that of either component alone or of the additive activity that would be predicted for the combinations based on the activities of the individual components. Thus in a potentiating ratio, the individual components act synergistically.

Synergism may be defined using a number of methods. For example, synergism may be defined as an RI of greater than unity using the method of Kern as modified by Romaneli (1998a, 1998b). The RI may be calculated as the ratio of expected cell survival (Sₑₓₚ, defined as the product of the survival observed with drug A alone and the survival observed with drug B alone) to the observed cell survival (S_{obs}) for the combination of A and B (RI=Sₑₓₚ/S_{obs}). Synergism may then be defined as an RI of greater than unity.

In another method, synergism may be determined by calculating the combination index (CI) according to the method of Chou and Talalay. CI values of 1, <1, and >1 indicate additive, synergistic and antagonistic effects respectively.

In a preferred embodiment of the invention, the c-FLIP inhibitor and the chemotherapeutic agent are present in concentrations sufficient to produce a CI of less than 1, preferably less than 0.85.

Synergism is preferably defined as an RI of greater than unity using the method of Kern as modified by Romaneli (1998a,b)). The RI may be calculated as the ratio of expected cell survival (Sₑₓₚ, defined as the product of the survival observed with drug A alone and the survival observed with drug B alone) to the observed cell survival (S_{obs}) for the combination of A and B (RI=Sₑₓₚ/S_{obs}). Synergism may then be defined as an RI of greater than unity.

In preferred embodiments of the invention, said specific binding member and chemotherapeutic agent are provided in concentrations sufficient to produce an RI of greater than 1.5, more preferably greater than 2.0, most preferably greater than 2.25.

The combined medicament thus preferably produces a synergistic effect when used to treat tumour cells.

The invention according to any of the first, second third, fourth and fifth aspect of the invention may be used for the killing of any cancer cell having a p53 mutation. The mutation may partially or totally inactivate p53 in a cell. In one embodiment of the invention, the p53 mutation is a p53 mutation, which totally inactivates p53. In another embodiment, the p53 mutation is a missense mutation resulting in the substitution of histidine (R175H mutation). In another embodiment, the p53 mutation is a missense mutation resulting in the substitution of tryptophan (R248W mutation) for arginine.

As described in the Examples, as well as testing the cytotoxicity of combinations of c-FLIP inhibitors and chemotherapeutic agents on cancer cells, the inventors further tested the effects of c-FLIP alone. The inventors unexpectedly observed that relatively potent inhibition of cFLIP using high concentrations of siRNA triggered apoptosis in the absence of chemotherapy in both RKO and H630 cell lines. This demonstration that cFLIP inhibition in the absence of chemotherapy is sufficient to trigger apoptosis in cancer cells enables the use of c-FLIP inhibition aole as a chemotherapeutic strategy.

Accordingly, in a sixth aspect of the invention, there is provided a method of killing cancer cells, comprising administration to said cells of an effective amount of a c-FLIP inhibitor, wherein the c-FLIP inhibitor is administered as the sole cytotoxic agent in the substantial absence of other cytotoxic agents.

A seventh aspect of the invention provides a method of treating cancer comprising administration to a subject in need thereof a therapeutically effective amount of a c-FLIP inhibitor, wherein the c-FLIP inhibitor is administered as the sole cytotoxic agent in the substantial absence of other cytotoxic agents.

An eighth aspect provides the use of a c-FLIP inhibitor as the sole cytotoxic agent in the preparation of a medicament for treating cancer, wherein the medicament is for treatment in the substantial absence of other cytotoxic agents.

A ninth aspect provides a pharmaceutical composition for the treatment of cancer, wherein the composition comprises a c-FLIP inhibitor as the sole cytotoxic agent and a pharmaceutically acceptable excipient, diluent or carrier, wherein the composition is for treatment in the absence of other cytotoxic agents.

The sixth to ninth aspects of the invention may be used in the treatment of any cancer. The cancer cells may comprise a p53 wild type genotype or, alternatively, may comprise p53 mutant genotypes. The mutation may partially or totally inactivate p53 in a cell. In one embodiment of the invention, the p53 mutation is a p53 mutation, which totally inactivates p53. In another embodiment, the p53 mutation is a missense mutation resulting in the substitution of histidine (R175H mutation). In another embodiment, the p53 mutation is a missense mutation resulting in the substitution of tryptophan (R248W mutation) for arginine.

Any suitable c-FLIP inhibitor may be used in methods of the invention. The inhibitor may be peptide or non-peptide.

In one preferred embodiment, said c-FLIP inhibitor is an antisense molecule which modulates the expression of the gene encoding c-FLIP.

In a more preferred embodiment, said c-FLIP inhibitor is an RNAi agent, which modulates expression of the c-FLIP gene. The agent may be an siRNA, an shRNA, a ddRNAi construct or a transcription template thereof, e.g., a DNA encoding an shRNA. In preferred embodiments the RNAi agent is an siRNA which is homologous to a part of the mRNA sequence of the gene encoding c-FLIP.

Preferred RNAi agents of and for use in the invention are between 15 and 25 nucleotides in length, preferably between 19 and 22 nucleotides, most preferably 21 nucleotides in length. In particularly preferred embodiments of the invention, the RNAi agent has the nucleotide sequence shown as SEQ ID NO: 1.

| | |
|---|---|
| AAG CAG TCT GTT CAA GGA GCA | (SEQ ID NO: 1) |
| or | |
| AAG GAA CAG CTT GGC GCT CAA. | (SEQ ID NO: 2) |

In another particularly preferred embodiment of the invention, the RNAi agent has the nucleotide sequence shown as SEQ ID NO: 2

| | |
|---|---|
| AAG CAG TCT GTT CAA GGA GCA | (SEQ ID NO: 1) |
| or | |
| AAG GAA CAG CTT GGC GCT CAA. | (SEQ ID NO: 2) |

Indeed such RNAi agents represents a tenth and eleventh independent aspects of the present invention.

According to a further aspect of the invention, there is provided a vector comprising the RNAi agent of the tenth aspect of the invention.

In a further aspect, there is provided a kit for the treatment of cancer associated with a p53 mutation, said kit comprising

(a) a c-FLIP inhibitor and
(b) a chemotherapeutic agent, wherein the chemotherapeutic agent is a thymidylate synthase inhibitor, a platinum cytotoxic agent or a topoisomerase inhibitor and
(c) instructions for the administration of (a) and (b) separately, sequentially or simultaneously.

Preferred features of each aspect of the invention are as for each of the other aspects mutatis mutandis unless the context demands otherwise.

### DETAILED DESCRIPTION

As described above, the present invention relates to methods of treatment of cancer, involving cFLIP inhibition.

The methods of the invention may involve the determination of expression of FLIP protein.

The expression of FLIP may be measured using any technique known in the art. Either mRNA or protein can be measured as a means of determining up- or down regulation of expression of a gene. Quantitative techniques are preferred. However semi-quantitative or qualitative techniques can also be used. Suitable techniques for measuring gene products include, but are not limited to, SAGE analysis, DNA microarray analysis, Northern blot, Western blot, immunocytochemical analysis, and ELISA.

RNA can be detected using any of the known techniques in the art. Preferably an amplification step is used as the amount of RNA from the sample may be very small. Suitable techniques may include real-time RT-PCR, hybridisation of copy mRNA (cRNA) to an array of nucleic acid probes and Northern Blotting.

For example, when using mRNA detection, the method may be carried out by converting the isolated mRNA to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of gene expression products of one or more of the genes encoding FLIP protein. Analysis may be accomplished using Southern Blot analysis to detect the presence of the gene products in the amplification product. Southern Blot analysis is known in the art. The analysis step may be further accomplished by quantitatively detecting the presence of such gene products in the amplification products, and comparing the quantity of product detected against a panel of expected values for known presence or absence in normal and malignant tissue derived using similar primers.

In e.g. determining gene expression in carrying out conventional molecular biological, microbiological and recombinant DNA techniques known in the art may be employed. Details of such techniques are described in, for example, Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992).

### Binding Members

In the context of the present invention, a “binding member” is a molecule which has binding specificity for another molecule, in particular a receptor, preferably a death receptor. The binding member may be a member of a pair of specific binding members. The members of a binding pair may be naturally derived or wholly or partially synthetically produced. One member of the pair of molecules may have an area on its surface, which may be a protrusion or a cavity, which specifically binds to and is therefore complementary to a particular spatial and polar organisation of the other member of the pair of molecules. Thus, the members of the pair have the property of binding specifically to each other. A binding member of the invention and for use in the invention may be any moiety, for example an antibody or ligand, which preferably can bind to a death receptor.

The binding member may bind to any death receptor. Death receptors include, Fas, TNFR, DR-3, DR-4 and DR-5. In preferred embodiments of the invention, the death receptor is FAS.

In preferred embodiments, the binding member comprises at least one human constant region.

### Antibodies

An “antibody” is an immunoglobulin, whether natural or partly or wholly synthetically produced. The term also covers any polypeptide, protein or peptide having a binding domain which is, or is homologous to, an antibody binding domain. These can be derived from natural sources, or they may be partly or wholly synthetically produced. Examples of antibodies are the immunoglobulin isotypes and their isotypic subclasses and fragments which comprise an antigen binding domain such as Fab, scFv, Fv, dAb, Fd; and diabodies.

A binding member for use in certain embodiments, the invention may be an antibody such as a monoclonal or polyclonal antibody, or a fragment thereof. The constant region of the antibody may be of any class including, but not limited to, human classes IgG, IgA, IgM, IgD and IgE. The antibody may belong to any sub class e.g. IgG1, IgG2, IgG3 and IgG4. IgG1 is preferred.

As antibodies can be modified in a number of ways, the term “antibody” should be construed as covering any binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

Examples of such fragments which can be used in the invention include the Fab fragment, the Fd fragment, the Fv fragment, the dAb fragment (Ward, E. S. et al., Nature 341:544-546 (1989)), F(ab′)2 fragments, single chain Fv molecules (scFv), bispecific single chain Fv diners (PCT/US92/09965) and “diabodies”, multivalent or multispecific fragments constructed by gene fusion (WO94/13804; P. Hollinger et al., Proc. Natl. Acad. Sci. USA 90:6444-6448 (1993)).

A fragment of an antibody or of a polypeptide for use in the present invention generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having death receptor, e.g. FAS neutralisation and/or binding activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

In preferred embodiments, the binding member is humanised. Methods for making humanised antibodies are known in the art e.g see U.S. Pat. No. 5,225,539. A humanised antibody may be a modified antibody having the hypervariable region of a monoclonal antibody and the constant region of a human antibody. Thus the binding member may comprise a human constant region. The variable region other than the hypervariable region may also be derived from the variable region of a human antibody and/or may also be derived from a monoclonal antibody. In such case, the entire variable region may be derived from murine monoclonal antibody and the antibody is said to be chimerised. Methods for making chimerised antibodies are known in the art (e.g see U.S. Pat. Nos. 4,816,397 and 4,816,567).

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

A typical antibody for use in the present invention is a humanised equivalent of CH11 or any chimerised equivalent of an antibody that can bind to the FAS receptor and any alternative antibodies directed at the FAS receptor that have been chimerised and can be use in the treatment of humans. Furthermore, the typical antibody is any antibody that can cross-react with the extracellular portion of the FAS receptor and either bind with high affinity to the FAS receptor, be internalised with the FAS receptor or trigger signalling through the FAS receptor.

### Production of Binding Members

Binding members, which may be used in certain aspects of the present invention may be generated wholly or partly by chemical synthesis. The binding members can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Another convenient way of producing a binding member suitable for use in the present invention is to express nucleic acid encoding it, by use of nucleic acid in an expression system. Thus the present invention further provides the use of (a) nucleic acid encoding a specific binding member which binds to a cell death receptor and (b) a chemotherapeutic agent and (c) a cFLIP inhibitor in the preparation of a medicament for treating cancer associated with a p53 mutation.

Nucleic acids of and/or for use in accordance with the present invention may comprise DNA or RNA and may be wholly or partially synthetic. In a preferred aspect, nucleic acid for use in the invention codes for a binding member of the invention as defined above. The skilled person will be able to determine substitutions, deletions and/or additions to such nucleic acids which will still provide a binding member suitable for use in the present invention.

Nucleic acid sequences encoding a binding member for use with the present invention can be readily prepared by the skilled person using the information and references contained herein and techniques known in the art (for example, see Sambrook, Fritsch and Maniatis, “Molecular Cloning”, A Laboratory Manual, Cold Spring Harbor Laboratory Press, 1989, and Ausubel et al, Short Protocols in Molecular Biology, John Wiley and Sons, 1992), given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding antibody fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

The nucleic acid may be comprised as construct(s) in the form of a plasmid, vector, transcription or expression cassette which comprises at least one nucleic acid as described above. The construct may be comprised within a recombinant host cell which comprises one or more constructs as above. Expression may conveniently be achieved by culturing under appropriate conditions recombinant host cells containing the nucleic acid. Following production by expression a specific binding member may be isolated and/or purified using any suitable technique, then used as appropriate.

Binding members-encoding nucleic acid molecules and vectors for use in accordance with the present invention may be provided isolated and/or purified, e.g. from their natural environment, in substantially pure or homogeneous form, or, in the case of nucleic acid, free or substantially free of nucleic acid or genes of origin other than the sequence encoding a polypeptide with the required function.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antibody fragments in prokaryotic cells such as E. coli is well established in the art. For a review, see for example Plückthun, Bio/Technology 9:545-551 (1991). Expression in eukaryotic cells in culture is also available to those skilled in the art as an option for production of a binding member, see for recent review, for example Reff, Curr. Opinion Biotech. 4:573-576 (1993); Trill et al., Curr. Opinion Biotech. 6:553-560 (1995).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Sambrook et al., Molecular Cloning: A Laboratory Manual: 2nd Edition, Cold Spring Harbor Laboratory Press (1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. eds., Short Protocols in Molecular Biology, 2nd Edition, John Wiley & Sons (1992).

The nucleic acid may be introduced into a host cell by any suitable means. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells under conditions for expression of the gene.

The nucleic acid may be integrated into the genome (e.g. chromosome) of the host cell. Integration may be promoted by inclusion of sequences which promote recombination with the genome in accordance with standard techniques. The nucleic acid may be on an extra-chromosomal vector within the cell, or otherwise identifiably heterologous or foreign to the cell.

### RNAi Agents

As described herein, c-FLIP inhibitors for use in the invention may be RNAi agents.

RNA interference (RNAi) or posttranscriptional gene silencing (PTGS) is a process whereby double-stranded RNA induces potent and specific gene silencing. RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger.

In one aspect, the invention provides methods of employing an RNAi agent to modulate expression, preferably reducing expression of a target gene, c-FLIP, in a mammalian, preferably human host. By reducing expression is meant that the level of expression of a target gene or coding sequence is reduced or inhibited by at least about 2-fold, usually by at least about 5-fold, e.g., 10-fold, 15-fold, 20-fold, 50-fold, 100-fold or more, as compared to a control. In certain embodiments, the expression of the target gene is reduced to such an extent that expression of the c-FLIP gene/coding sequence is effectively inhibited. By modulating expression of a target gene is meant altering, e.g., reducing, translation of a coding sequence, e.g., genomic DNA, mRNA etc., into a polypeptide, e.g., protein, product.

The RNAi agents that may be employed in preferred embodiments of the invention are small ribonucleic acid molecules (also referred to herein as interfering ribonucleic acids), that are present in duplex structures, e.g., two distinct oligoribonucleotides hybridized to each other or a single ribooligonucleotide that assumes a small hairpin formation to produce a duplex structure. Preferred oligoribonucleotides are ribonucleic acids of not greater than 100 nt in length, typically not greater than 75 nt in length. Where the RNA agent is an siRNA, the length of the duplex structure typically ranges from about 15 to 30 bp, usually from about 20 and 29 bps, most preferably 21 bp. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

In certain embodiments, instead of the RNAi agent being an interfering ribonucleic acid, e.g., an siRNA or shRNA as described above, the RNAi agent may encode an interfering ribonucleic acid. In these embodiments, the RNAi agent is typically a DNA that encodes the interfering ribonucleic acid. The DNA may be present in a vector.

The RNAi agent can be administered to the host using any suitable protocol known in the art. For example, the nucleic acids may be introduced into tissues or host cells by viral infection, microinjection, fusion of vesicles, particle bombardment, or hydrodynamic nucleic acid administration.

DNA directed RNA interference (ddRNAi) is an RNAi technique which may be used in the methods of the invention. ddRNAi is described in U.S. Pat. No. 6,573,099 and GB 2353282. ddRNAi is a method to trigger RNAi which involves the introduction of a DNA construct into a cell to trigger the production of double stranded (dsRNA), which is then cleaved into small interfering RNA (siRNA) as part of the RNAi process. ddRNAi expression vectors generally employ RNA polymerase III promoters (e.g. U6 or H1) for the expression of siRNA target sequences transfected in mammalian cells. siRNA target sequences generated from a ddRNAi expression cassette system can be directly cloned into a vector that does not contain a U6 promoter. Alternatively short single stranded DNA oligos containing the hairpin siRNA target sequence can be annealed and cloned into a vector downstream of the pol III promoter. The primary advantages of ddRNAi expression vectors is that they allow for long term interference effects and minimise the natural interferon response in cells.

### Antisense Nucleic Acids

As described herein, c-FLIP inhibitors for use in the invention may be anti-sense molecules or nucleic acid constructs that express such anti-sense molecules as RNA. The antisense molecules may be natural or synthetic. Synthetic antisense molecules may have chemical modifications from native nucleic acids. The antisense sequence is complementary to the mRNA of the targeted c-FLIP gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the c-FLIP sequence in an appropriate vector, where the transcriptional initiation is oriented such that an antisense strand is produced as an RNA molecule. Alternatively, the antisense molecule may be a synthetic oligonucleotide. Antisense oligonucleotides will generally be at least about 7, usually at least about 12, more usually at least about 16 nucleotides in length, and usually not more than about 50, preferably not more than about 35 nucleotides in length.

A specific region or regions of the endogenous c-FLIP sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorodiamidate linkages, methylphosphonates phosphorothioates; phosphorodithioates, where both of the non-bridging oxygens are substituted with sulfur; phosphoroamidites; alkyl phosphotriesters and boranophosphates. Achiral phosphate derivatives include 3′-O-5′-S-phosphorothioate, 3′-S-5′-O-phosphorothioate, 3′-CH2-5′-O-phosphonate and 3′-NH-5′-O-phosphoroamidate. Peptide nucleic acids may replace the entire ribose phosphodiester backbone with a peptide linkage. Sugar modifications may also be used to enhance stability and affinity.

### Chemotherapeutic Agents

Any suitable thymidylate synthase inhibitor, platinum cytotoxic agent or topoisomerase inhibitor may be used in the present invention. Examples of thymidylate synthase inhibitors which may be used in the methods of the invention include 5-FU, MTA and TDX. In a preferred embodiment, the thymidylate synthase inhibitor is 5-FU. Examples of platinum cytotoxic agents which may be used include cisplatin and oxaliplatin. In a particularly preferred embodiment of the invention, the chemotherapeutic agent is cisplatin. A topoisomerase inhibitor, which may be used in the present invention is irenotecan (CPT-11).

### Treatment

Treatment” includes any regime that can benefit a human or non-human animal. The treatment may be in respect of an existing condition or may be prophylactic (preventative treatment). Treatment may include curative, alleviation or prophylactic effects.

“Treatment of cancer” includes treatment of conditions caused by cancerous growth and includes the treatment of neoplastic growths or tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, cervical and ovarian carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, gliomas and retinoblastomas.

In preferred embodiments of the invention, the cancer is one or more of colorectal, breast, ovarian, cervical, gastric, lung, liver, skin and myeloid (e.g. bone marrow) cancer.

### Administration

As described above, c-FLIP inhibitors of and for use in the present invention may be administered in any suitable way. Moreover in any of the first to fifth aspects of the invention, they may be used in combination therapy with other treatments, for example, other chemotherapeutic agents or binding members. In such embodiments, the c-FLIP inhibitors or compositions of the invention may be administered simultaneously, separately or sequentially with another chemotherapeutic agent.

Where administered separately or sequentially, they may be administered within any suitable time period e.g. within 1, 2, 3, 6, 12, 24, 48 or 72 hours of each other. In preferred embodiments, they are administered within 6, preferably within 2, more preferably within 1, most preferably within 20 minutes of each other.

In a preferred embodiment, the c-FLIP inhibitors and/or compositions of the invention are administered as a pharmaceutical composition, which will generally comprise a suitable pharmaceutical excipient, diluent or carrier selected dependent on the intended route of administration.

The c-FLIP inhibitors and/or compositions of the invention may be administered to a patient in need of treatment via any suitable route.

Some suitable routes of administration include (but are not limited to) oral, rectal, nasal, topical (including buccal and sublingual), vaginal or parenteral (including subcutaneous, intramuscular, intravenous, intradermal, intrathecal and epidural) administration. Intravenous administration is preferred.

The c-FLIP inhibitor, product or composition may be administered in a localised manner to a tumour site or other desired site or may be delivered in a manner in which it targets tumour or other cells.

Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The c-FLIP inhibitors and/or compositions of the invention may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly(2-hydroxyethyl-methacrylate) or ethylene vinyl acetate (Langer et al, J. Biomed. Mater. Res. 15: 167-277, 1981, and Langer, Chem. Tech. 12:98-105, 1982). Liposomes containing the polypeptides are prepared by well-known methods: DE 3,218, 121A; Epstein et al, PNAS USA, 82: 3688-3692, 1985; Hwang et al, PNAS USA, 77: 4030-4034, 1980; EP-A-0052522; E-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage.

Examples of the techniques and protocols mentioned above and other techniques and protocols which may be used in accordance with the invention can be found in Remington's Pharmaceutical Sciences, 16th edition, Oslo, A. (ed), 1980.

### Pharmaceutical Compositions

Pharmaceutical compositions according to the present invention, and for use in accordance with the present invention may comprise, in addition to active ingredients, a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

The formulation may be a liquid, for example, a physiologic salt solution containing non-phosphate buffer at pH 6.8-7.6, or a lyophilised powder.

### Dose

The c-FLIP inhibitors or compositions of the invention are preferably administered to an individual in a “therapeutically effective amount”, this being sufficient to show benefit to the individual. The actual amount administered, and rate and time-course of administration, will depend on the nature and severity of what is being treated.

Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

### BRIEF DESCRIPTION OF THE FIGURES

The invention will now be described further in the following non-limiting examples. Reference is made to the accompanying drawings in which:

FIG. 1A illustrates Western blot analysis of Fas, FasL, procaspase 8, FADD, BID, Bcl-2, c-FLIP_{L}, c-FLIP_{S}, DcR3 and β-tubulin in MCF-7 cells 72 hours after treatment with 5 μM 5-FU and 50 nM TDX.

FIG. 1B illustrates analysis of the interaction between Fas and FasL following treatment with 5 μM 5-FU and 50 nM TDX for 48 hours. Lysates were immunoprecipitated using a FasL polyclonal antibody and analysed by Western blot using a Fas monoclonal antibody.

FIG. 1C illustrates analysis of the interaction between Fas and p43-c-FLIP_{L} following treatment with 5 μM 5-FU and 50 nM TDX for 48 hours. Lysates were immunoprecipitated using the anti-Fas CH-11 monoclonal antibody and analysed by Western blot using a c-FLIP monoclonal antibody.

FIG. 2A illustrates flow cytometry of MCF-7 cells treated with no drug (control), CH-11 alone (250 ng/ml), 5-FU alone (5 μM) for 96 hours, or co-treated with 5-FU for 72 hours followed by CH-11 for a further 24 hours.

FIG. 2B illustrates flow cytometry of MCF-7 cells treated with no drug (control), CH-11 alone (250 ng/ml), TDX alone (50 nM) for 96 hours, or co-treated with TDX for 72 hours followed by CH-11 for a further 24 hours.

FIG. 2C illustrates Western blot analysis of Fas expression in MCF-7 cells treated with 5 μM 5-FU for 48 hours. β-tubulin was assessed as a loading control.

FIG. 2D illustrates flow cytometry of MCF-7 cells treated with no drug (control), CH-11 alone (250 ng/ml), OXA alone (5 μM) for 96 hours, or co-treated with OXA for 72 hours followed by CH-11 for a further 24 hours.

FIG. 2E illustrates Western blot analysis of Fas, procaspase 8 and PARP expression in MCF-7 cells treated with 5 μM 5-FU alone for 96 hours, or co-treated with 5-FU for 72 hours followed by CH-11 for a further 24 hours.

FIG. 2F illustrates Western blot analysis examining the kinetics of caspase 8 activation and c-FLIP_{L} processing in MCF-7 cells treated for 72 hours with 5 μM 5-FU followed by 250 ng/ml CH-11 for the indicated times.

FIG. 3A illustrates Western blot analysis of Fas expression in HCT116 cells treated with 5-FU, TDX or OXA for 48 hours. Equal loading was assessed using a β-tubulin antibody.

FIG. 3B illustrates Western blot analysis of procaspase 8 and PARP expression in HCT116 cells treated no drug (Con), 5 μM 5-FU, 100 nM TDX or 2 μM OXA in the presence or absence of co-treatment with 200 ng/ml CH-11. For each combined treatment the cells were pre-treated with chemotherapeutic drug for 24 hours followed by CH-11 for a further 24 hours.

FIG. 4A illustrates Western blot of c-FLIP_{L} expression in MCF-7 cells stably transfected with a FLIPL (FL) construct or empty vector (EV).

FIG. 4B illustrates MTT cell viability assays in EV68, FL44 and FL64 cells treated with 5 μM 5-FU in combination with 250 ng/ml CH-11. The combined treatment resulted in a synergistic decrease in cell viability in EV68 cells (RI=2.06), but not FL44 (RI=1.14) or FL64 (1.01) cells.

FIG. 4C illustrates Western blot analysis of c-FLIP_{L}, procaspase 8 and PARP expression in EV68 and FL64 cells treated with no drug (Con) or 5 μM 5-FU in the presence (+) or absence (−) of co-treatment with 250 ng/ml CH-11. For each combined treatment, the cells were pre-treated with 5-FU for 72 hours followed by CH-11 for a further 24 hours.

FIG. 5A illustrates MTT cell viability assays in EV68, FL44 and FL64 cells treated with 50 nM TDX or 500 nM MTA in the presence and absence of 250 ng/ml CH-11. Combined TDX/CH-11 treatment resulted in a synergistic decrease in cell viability in EV68 cells (RI=1.75), that was significantly reduced in FL44 (RI=1.22) or FL64 (RI=1.19) cells. Combined MTA/CH-11 treatment resulted in a synergistic decrease in cell viability in EV68 cells (RI=1.86), that was significantly reduced in FL44 (RI=1.29) and FL64 (RI=1.06) cells.

FIG. 5B illustrates MTT cell viability assays in EV68, FL44 and FL64 cells treated with 2.54M OXA in the presence and absence of 250 ng/ml CH-11. Combined OXA/CH-11 treatment resulted in a synergistic decrease in cell viability in EV68 cells (RI=2.13), that was significantly reduced in FL64 (RI=1.22) or FL44 (1.19) cells.

FIG. 5C Western blot analysis of procaspase 8 and PARP expression in EV68 and FL64 cells treated with 50 nM TDX or 500 nM MTA in the presence (+) or absence (−) of co-treatment with 250 ng/ml CH-11.

FIG. 5D illustrates Western blot analysis of procaspase 8 and PARP expression in EV68 and FL64 cells treated with 2.5 μM OXA in the presence (+) or absence (−) of co-treatment with 250 ng/ml CH-11. For each combined treatment, the cells were pre-treated with 5-FU for 72 hours followed by CH-11 for a further 24 hours.

FIG. 6A illustrates c-FLIP_{L} and c-FLIP_{S} expression in HCT116 cells transfected with 0, 1 and 10 nM FLIP-targeted siRNA for 48 hours. Equal loading was assessed using a β-tubulin antibody.

FIG. 6B illustrates MTT cell viability assays of HCT116 cells transfected with 5 nM FLIP-targeted (FT) or scrambled control (SC) siRNA in the presence and absence of co-treatment with 5 μM 5-FU. Combined treatment with 5-FU and FT siRNA resulted in a synergistic decrease in cell viability (RI=1.92, p<0.0005). No synergistic decrease in viability was observed in cells co-treated with 5-FU and SC siRNA (RI=0.98).

FIG. 6C illustrates Western blot analysis of caspase 8 activation and PARP cleavage in HCT116 cells 48 hours after treatment with no drug, 5 μM 5-FU or 100 nM TDX in mock transfected cells (M), cells transfected with 1 nM scrambled control (SC) and cells transfected with 1 nM FLIP-targeted (FT) siRNA.

FIG. 7A illustrates c-FLIP_{L} and c-FLIP_{S} expression in MCF-7 cells transfected with 10 nM FLIP-targeted (FT) or scrambled control (SC) siRNA for 48 hours. Equal loading was assessed using a β-tubulin antibody.

FIG. 7B illustrates MTT cell viability assays of MCF-7 cells transfected with 2.5 nM FT siRNA in the presence and absence of co-treatment with 5 μM 5-FU. The combined treatment resulted in a synergistic decrease in cell viability (RI=1.56, p<0.005). FIG. 7C Western blot analysis of PARP cleavage in MCF-7 cells 96 hours after treatment with 5-FU in the presence (+) and absence (−) of 10 nM FLIP-targeted siRNA.

FIG. 8 illustrates MTT cell viability assays of HCT116 cells transfected with 0.5 nM FT or SC siRNA in the presence and absence of co-treatment with: FIG. 8A 5 μM 5-FU; FIG. 8B 100 nM TDX and FIG. 8C 1 μM OXA. Cells were assayed after 72 hours. Combined treatment with FT siRNA (but not SC siRNA) and each cytotoxic drug resulted in synergistic decreases in cell viability as indicated by the RI values (p<0.0005 for each combination).

FIG. 9 illustrates: A Western blot analysis of Fas expression in p53 wild type HCT116 cells treated with 5-FU or oxaliplatin (OXA) for 48 hours. B Western blot analysis of caspase 8 activation, PARP cleavage and c-FLIP expression in p53 wild type HCT116 cells treated with no drug (Con), 5 μM 5-FU, or 1 μM OXA in the presence or absence of co-treatment with 200 ng/mL CH-11. For each combined treatment the cells were pre-treated with chemotherapeutic drug for 24 hours followed by CH-11 for a further 24 hours.

FIG. 10 illustrates: A c-FLIP_{L} and c-FLIP_{S} expression in HLacZ, HFL17, HFL24, HFS19 and HFS44 cell lines. B Flow cytometric analysis of cell cycle arrest and apoptosis in HLacZ, HFL17, HFL24, HFS19 and HFS44 cell lines 72 hours after treatment with 5 μM 5-FU, 1 μM oxaliplatin. (OXA) and 5 μM CPT-11. C Flow cytometric analysis of HLacZ, HFL17, HFL24, HFS19 and HFS44 cells after co-treatment with 50 ng/mL CH-11 and 2.5 μM 5-FU, 0.5 μM oxaliplatin (OXA) and 1 μM CPT-11. For each combined treatment the cells were pre-treated with chemotherapeutic drug for 24 hours followed by CH-11 for a further 24 hours.

FIG. 11 illustrates: A c-FLIP_{L} and c-FLIP_{S} expression in p53 wild type HCT116 cells transfected with 1 nM control siRNA (SC) and 1 nM FLIP-targeted (FT) siRNA for 24 hours. B Flow cytometric analysis of apoptosis in HCT116 cells transfected with 0.5 nM FT or 0.5 nM SC siRNA. Transfected cells were co-treated with no drug, 5 μM 5-FU, or 1 μm oxaliplatin (OXA) for 48 hours. C (Panel 1) Western blot analysis of caspase 8 activation and PARP cleavage in HCT116 cells 48 hours after treatment of mock transfected cells (M), cells transfected with 0.5 nM SC and cells transfected with 0.5 nM FT siRNA with no drug, 5 μM 5-FU or 100 nM TDX. (Panel 2) Caspase 8 activation and PARP cleavage in HCT116 cells transfected with 0.5 nM SC or 0.5 nM FT siRNA and treated with no drug, or 1 μM oxaliplatin (OXA) for 24 hours. (Panel 3) Caspase 8 activation and PARP cleavage in HCT116 cells after transfection with 0.5 nM SC or 0.5 nM FT siRNA and treatment with no drug, 2.5 μM or 5 μM CPT-11 for 24 hours. D MTT cell viability assays in HCT116p53^{+/+} cells transfected with FT siRNA and co-treated with 5-FU, oxaliplatin (OXA) and CPT-11. Cell viability was assayed after 72 hours. The nature of the interaction between the chemotherapeutic drugs and FT siRNA was determined by calculating the combination index (CI) according to the method of Chou and Talalay. CI values of 1, <1, and >1 indicate additive, synergistic and antagonistic effects respectively. Results are representative of at least 3 separate experiments.

FIG. 12 illustrates: A Western blot analysis of c-FLIP_{L} and c-FLIP_{S} expression in p53 wild type (wt) and null HCT116 cells. B Western blot analysis of c-FLIP_{L} and c-FLIP_{S} expression in HCT116p53^{-/-} cells transfected with 1 nM control siRNA (SC) and 1 nM FLIP-targeted (FT) siRNA for 24 hours. C Flow cytometric analysis of apoptosis in HCT116p53^{-/-} cells transfected with 1 nM FT or 1 nM SC siRNA. Transfected cells were co-treated with no drug, 5 μM 5-FU, 5 μM oxaliplatin (OXA) or 1 μM CPT-11 for 72 hours. D MTT cell viability assays in HCT116p53^{-/-} cells transfected with FT siRNA and co-treated with 5-FU, oxaliplatin (OXA), and CPT-11. Cell viability was assayed after 72 hours. The nature of the interaction between the chemotherapeutic drugs and FLIP-targeted siRNAs was determined by calculating the combination index (CI) according to the method of Chou and Talalay. Results are representative of at least 3 separate experiments.

FIG. 13 illustrates: A c-FLIP_{L} and c-FLIP_{S} expression in RKO and H630 cells transfected with 1 nM control siRNA (SC) and 1 nM FLIP-targeted (FT) siRNA for 24 hours. B Flow cytometric analysis of apoptosis in RKO cells transfected with 2.5 mM FT or 2.5 nM SC siRNA and H630 cells transfected with 1 nM FT or 1 nM SC siRNA. SiRNA-transfected RKO cells were co-treated with no drug, 5 μM 5-FU, 1 μM oxaliplatin (OXA) or 2.5 μM CPT-11 for 72 hours. SiRNA-transfected H630 cells were co-treated with no drug, 5 μM 5-FU, 2.5 μM oxaliplatin (OXA) or 1 μM CPT-11 for 72 hours. C MTT cell viability assays in RKO and H630 cells transfected with FT siRNA and co-treated with 5-FU, oxaliplatin (OXA), and CPT-11. Cell viability was assayed after 72 hours. The nature of the interaction between the chemotherapeutic drugs and FLIP-targeted siRNAs was determined by calculating the combination index (CI) according to the method of Chou and Talalay. Results are representative of at least 3 separate experiments.

FIG. 14 illustrates: A MTT cell viability assays in HCT116p53^{+/+} cells transfected with FT or SC siRNA for 72 hours. B Western blot analysis of c-FLIP expression and PARP cleavage in p53 wild type (p53^{+/+}) and p53 null (p53^{-/-}) HCT116 cells 24 hours after transfection with 0, 1 and 10 nM FT siRNA. C Flow cytometric analysis of apoptosis in p53 wild type (p53^{+/+}) and p53 null (p53^{-/-}) HCT116 cells transfected with FT or SC siRNA for 48 hours. D Apoptosis in HCT116p53^{-/-} cells transfected with FT siRNA for 48 and 72 hours. E Apoptosis in RKO cells transfected with FT or SC siRNA for 72 hours. F Apoptosis in H630 cells transfected with FT or SC siRNA for 72 hours.

FIG. 15 illustrates: A Kinetics of c-FLIP down-regulation, caspase 8 activation and PARP cleavage in HCT116p53^{+/+} cells transfected with 0, 1 and 10 nM FT siRNA. B Flow cytometric analysis of the kinetics of apoptosis induction in HCT116p53^{+/+} cells transfected with 10 nM FT or 10 nM SC siRNA.

FIG. 16 illustrates: A c-FLIP_{L} and c-FLIP_{S} expression and PARP cleavage in p53 wild type HCT116 cells transfected with 10 nM control siRNA (SC) and 10 nM FLIP_{L}-specific (FL) siRNA for 24 hours. B Western blot analysis of PARP cleavage in HCT116 cells transfected with 0.5 nM SC or 0.5 nM FL siRNA and treated with no drug, 1 μM oxaliplatin (OXA) or 2.5 μM for 24 hours, or 5 μM 5-FU for 48 hours. C MTT cell viability assays in HCT116p53^{+/+} cells transfected with FL siRNA and co-treated with 5-FU oxaliplatin (OXA), and CPT-11. Cell viability was assayed after 72 hours. The nature of the interaction between the chemotherapeutic drugs and FLIP-targeted siRNAs was determined by calculating the combination index (CI) according to the method of Chou and Talalay. Results are representative of at least 3 separate experiments.

FIG. 17 illustrates graphs of RI values calculated from MTT cell viability assays of the chemotherapeutic agents 5-FU, Tomudex (TDX), CPT-11 and Oxaliplatin used in combination with the agonistic anti-Fas antibody CH-11 (200 ng/ml). The RI is calculated as ratio of the expected cell survival (Sₑₓₚ, defined as the product of the survival observed with drug A alone and the survival observed with drug B alone) to the observed cell survival (S_{obs}) for the combination of A and B (RI=Sₑₓₚ/S_{obs}). Synergism is defined as an RI greater than 1.

FIG. 18 illustrates A, Flow cytometry analysis of cells stained with propidium iodide stained HCT116 p53 wild-type and null cells treated with 5-FU (5 μM), TDX (50 nM), CPT-11 (5 μM) and Oxaliplatin (1 μM) for 24 hours and then with CH-11 (50 ng/ml) for an additional 24 hours. B, Sub G0/G1 populations for the HCT116p53 wild-type and null cell lines treated with chemotherapy drugs with and without CH-11 50 ng/ml.

FIG. 19 illustrates the effect of adding CH-11 200 ng/ml for 24 hours to HCT116 p53 wild-type and null cells already treated for 24 hours with 5-FU (5 μM), CPT-11 (5 μM) and Oxaliplatin (1 μM) on PARP cleavage and activation of procaspase 8 by Western blot analysis.

### EXAMPLES

### Materials and Methods

Cell Culture. All cells were maintained in 5% CO₂ at 37° C. MCF-7 cells were maintained in DMEM with 10% dialyzed bovine calf serum supplemented with 1 mM sodium pyruvate, 2 mM L-glutamine and 50 μg/ml penicillin/streptomycin (from Life Technologies Inc., Paisley, Scotland). HCT116p53^{+/+} and HCT116p53^{-/-} isogenic human colorectal cancer cells were kindly provided by Professor Bert Vogelstein (John Hopkins University, Baltimore, Md.). HCT116 cells were grown in McCoy's 5A medium (GIBCO) supplemented with 10% dialysed foetal calf serum, 50 mg/ml penicillin-streptomycin, 2 mM L-glutamine and 1 mM sodium pyruvate. Stably transfected MCF-7 and HCT116 cell lines and mixed populations' of transfected cells were maintained in medium supplemented with 100 μg/ml (MCF-7) or 1.5 mg/ml (HCT116) G418 (from Life Technologies Inc).

Generation of c-FLIP overexpressing cell lines. c-FLIP_{L} and c-FLIP_{S} coding regions were PCR amplified and ligated into the pcDNA/V5-His TOPO vector according to the manufacturer's instructions (Life Technologies Inc.). HCT116p53^{+/+} cells were co-transfected with 10 μg of each c-FLIP expression construct and 1 μg of a construct expressing a puromycin resistance gene (pIRESpuro3, Clontech) using GeneJuice. Stably transfected HCT116 cells were selected and maintained in medium supplemented with 1 μg/ml puromycin (Life Technologies Inc.). Stable overexpression of c-FLIP was assessed by Western blot analysis.

Western Blotting. Western blots were performed as previously described (Longley et al., 2002). The Fas/CD95, Bcl-2 and BID (Santa Cruz Biotechnology, Santa Cruz, Calif.), caspase 8 (Oncogene Research Products, Darmstadt, Germany), PARP (Pharmingen, BD Biosciences, Oxford, England), c-FLIP (NF-6, Alexis, Bingham UK) DcR3 (Imgenex, San Diego, Calif.) mouse monoclonal antibodies were used in conjunction with a horseradish peroxidase (HRP)-conjugated sheep anti-mouse secondary antibody (Amersham, Little Chalfont, Buckinghamshire, England). FasL rabbit polyclonal antibody (Santa Cruz Biotechnology) was used in conjunction with an HRP-conjugated donkey anti-rabbit secondary antibody (Amersham). Equal loading was assessed using a β-tubulin mouse monoclonal primary antibody (Sigma).

Co-immunoprecipitation reactions. 250 μl of Protein A (IgG) or Protein L (IgM) Sepharose beads (Sigma) and 1 μg of the appropriate antibody were mixed at 4° C. for 1 hour. Antibody-associated beads were washed three times with ELB buffer (250 mM NaCl, 0.1% IPEGAL, 5 mM EDTA, 0.5 mM DTT, 50 mM HEPES). Protein lysate (200-400 μg) was then added, and the mixture rotated at 4° C. for 1 hour. The beads were then washed in ELB buffer five times and resuspended in 100 μl of Western sample buffer (250 mM TRIS pH 6.8, 4% SDS, 2% glycerol, 0.02% bromophenol blue) containing 10% β-mercaptoethanol. The samples were then heated at 95° C. for 5 minutes and centrifuged (5 mins/4,000 rpm/4° C.). The supernatant was collected and analysed by Western blotting.

Cell Viability Assays. Cell viability was assessed by MTT (3-(4,5-dimethylthiazol-2-yl)-2,5-diphenyltetrazolium bromide, Sigma) assay (Mosmann, 1983). To investigate drug-induced Fas-mediated apoptosis, cells were seeded at 2,000-5,000 cells per well on 96-well plates. After 24 hours, the cells were treated with a range of concentrations of 5-FU, TDX, MTA or OXA for 24-72 hours followed by the agonistic Fas monoclonal antibody, CH-11 (MBL, Watertown, Mass.) for a further 24-48 hours. To assess chemotherapy/siRNA interactions, 20,000-50,000 cells were seeded per well on 24-well plates. Twenty-four hours later, the cells were transfected with FLIP-targeted (FT) or scrambled siRNA (SC). Four hours after transfection, the cells were treated with a range of concentrations of each drug for a further 72-96 hours. MTT (0.5 mg/ml) was added to each well and the cells were incubated at 37° C. for a further 2 hours. The culture medium was removed and formazan crystals reabsorbed in 200 μl (96-well) or 1 ml (24-well) DMSO. Cell viability was determined by reading the absorbance of each well at 570 nm using a microplate reader (Molecular Devices, Wokingham, England).

Flow Cytometric Analysis. Cells were seeded at 1×10⁵ per well of a 6-well tissue culture plate. After 24 hours, 5-FU, TDX or OXA was added to the medium and the cells cultured for a further 72 hours, after which time 250 ng/ml CH-11 was added for 24 hours. DNA content of harvested cells was evaluated after propidium iodide staining of cells using the EPICS XL Flow Cytometer (Coulter, Miami, Fla.).

siRNA transfections. FLIP-targeted siRNA was designed using the Ambion siRNA target finder and design tool (www.ambion.com/techlib/misc/siRNA_finder.html) to inhibit both splice variants of c-FLIP. Both c-FLIP-targeted (FT) and scrambled control (SC) siRNA were obtained from Xeragon (Germantown, Md.). The FT siRNA sequence used was: AAG CAG TCT GTT CAA GGA GCA. The FL siRNA sequence used was: AAG GAA CAG CTT GGC GCT CAA. The control non-silencing siRNA sequence (SC) used was: AAT TCT CCG AAC GTG TCA CGT. siRNA transfections were performed on sub-confluent cells incubated in Optimem medium using the oligofectamine reagent (both from Life Technologies Inc) according to the manufacturer's instructions.

Statistical Analyses. The nature of the interaction between the chemotherapeutic drugs and FLIP-targeted siRNAs was determined by calculating the combination index (CI) according to the method of Chou and Talalay (14). CI values were calculated from isobolograms generated using the CalcuSyn software programme (Microsoft Windows). According to the definitions of Chou and Talalay, a CI value of 0.85-0.9 is slightly synergistic, 0.7-0.85 is moderately synergistic, 0.3-0.7 is synergistic and 0.1-0.3 is strongly synergistic. An unpaired two-tailed t test was used to determine the significance of changes in levels of apoptosis between different treatment groups.

### Results

### Example 1

### c-FLIP_{L} is Up-Regulated, Processed and Bound to Fas in Response to 5-FU and TDX

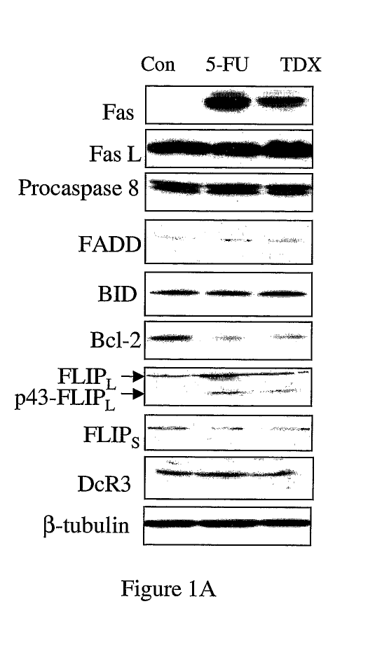

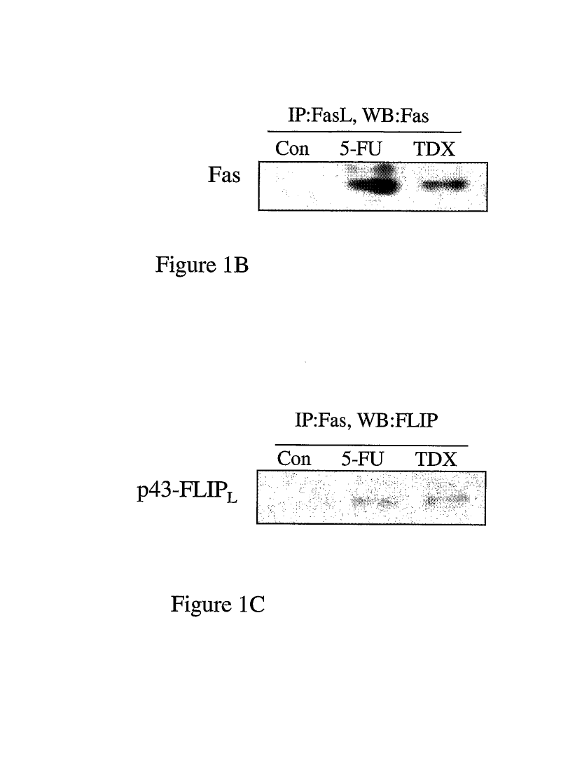

Analysis of Fas expression in MCF-7 cells revealed that it was up-regulated by ˜12-fold 72 hours after treatment with an IC60 dose 5-FU and was also highly up-regulated (by ˜7-fold) in response to treatment with an IC60 dose (25 nM) of TDX (FIG. 1A). FasL expression was unaffected by each drug treatment, but appeared to be highly expressed in these cells. Expression of FADD was also unaffected by drug treatment. Somewhat surprisingly, neither caspase 8, nor its substrate BID were activated in 5-FU- or TDX-treated cells as indicated by a lack of down-regulation of the levels of procaspase 8 or full-length BID (FIG. 1A). Bcl-2 was highly down-regulated in response to each agent. Interestingly, c-FLIP_{L} but not c-FLIP_{S} was up-regulated by drug treatment. Furthermore, c-FLIP_{L} was processed to its p43-form indicative of its recruitment and processing at the DISC (FIG. 1A). Expression of the Fas decoy receptor DcR3 was unaltered by drug treatment in these cells.

To further investigate the apparent inhibition of capsase 8 activation in 5-FU- and TDX-treated cells, we analysed the interaction between Fas and FasL following drug treatment. Co-immunoprecipitation reactions demonstrated that there was increased Fas-FasL binding following drug treatment (FIG. 1B), suggesting that the inhibition of caspase 8 activation was occurring downstream of receptor ligation. In support of this, we found that drug treatment increased the interaction between Fas and p43-c-FLIP_{L} (FIG. 1C). These results suggested the involvement of c-FLIP_{L} in inhibiting drug-induced activation of Fas-mediated apoptosis in MCF-7 cells.

### Example 2

### Activation of Drug-Induced Apoptosis by the Fas-Targeted Antibody CH-11 Coincides with Processing of c-FLIP_{L}

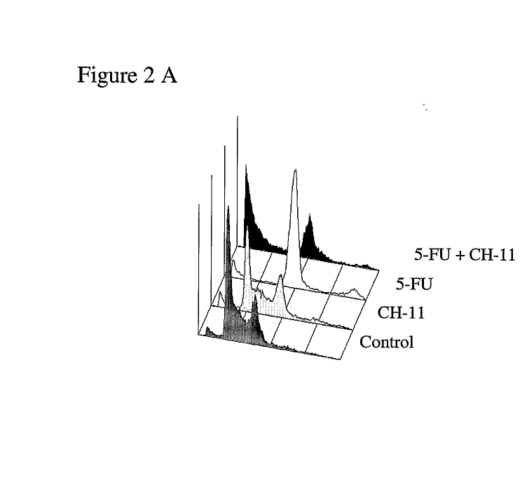

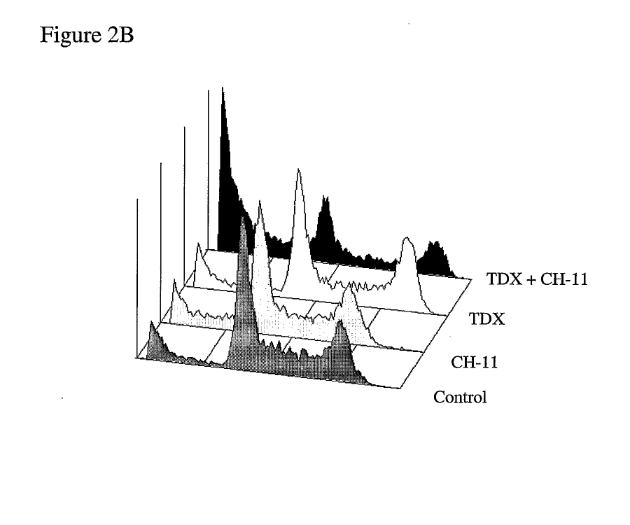

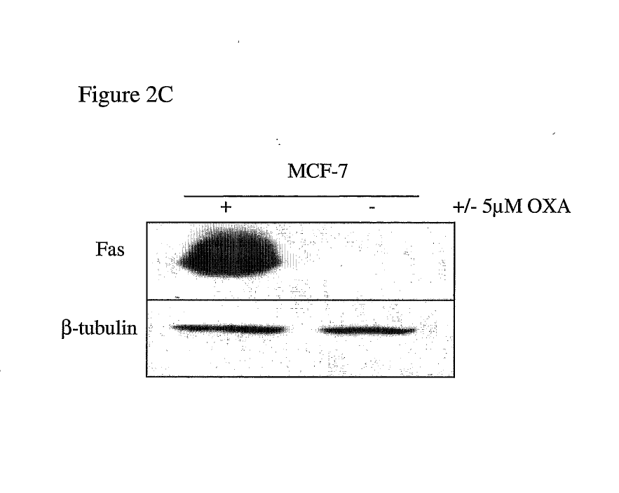

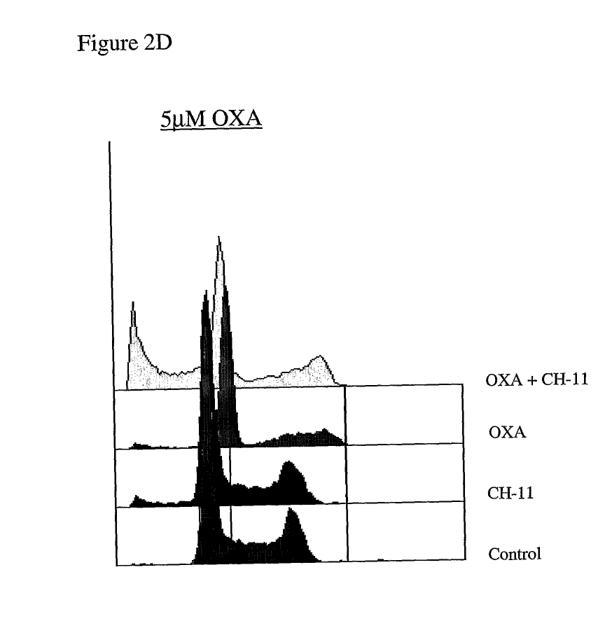

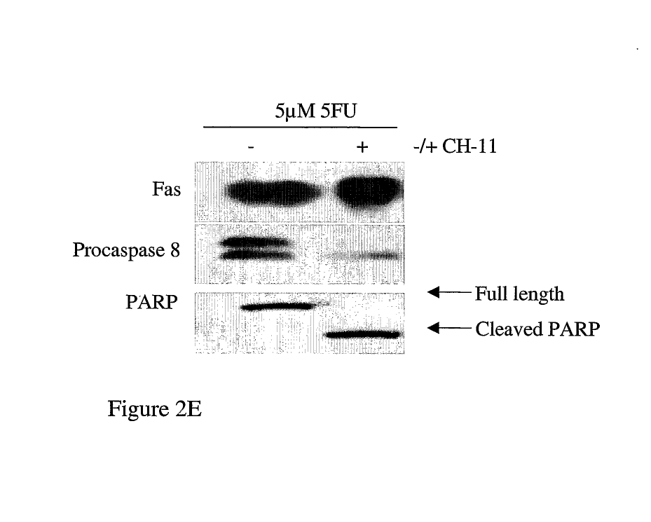

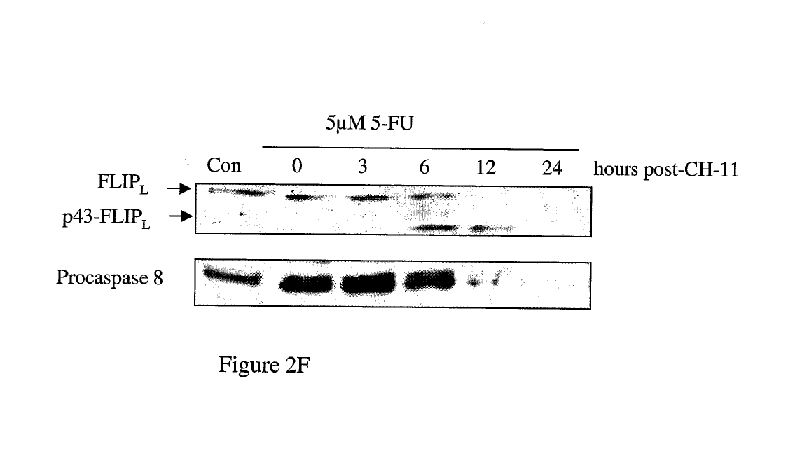

Expression of FasL by activated T cells and NK cells induces apoptosis of Fas expressing target cells in vivo. To mimic the effects of these immune effector cells in vitro, the agonistic Fas monoclonal antibody CH-11 was used. Cells were treated with either 5-FU or TDX for 72 hours followed by 250 ng/ml CH-11 treatment for 24 hours. We found that CH-11 alone had little effect on apoptosis (FIGS. 2A and B). Treatment with 5-FU alone for 96 hours resulted in a modest-2-fold induction of apoptosis in response to 5 μM 5-FU (FIG. 2A). However, addition of CH-11 to 5-FU-treated cells resulted in a dramatic increase in apoptosis, with a ˜55% of cells in the sub-G1/G0 apoptotic phase following co-treatment with 5 μM 5-FU and CH-11. Similarly, the combination of TDX with CH-11 resulted in dramatic activation of apoptosis, with ˜60% of cells in the sub-G1/G0 apoptotic phase following combined treatment with 25 nM TDX and CH-11 (FIG. 2B). We also examined the effect of CH-11 on apoptosis induced by the DNA-damaging agent OXA, which also potently induces Fas expression in MCF-7 cells (FIG. 2C). Similar to its effect on 5-FU and TDX-treated cells, CH-11 induced apoptosis of OXA-treated cells, with ˜50% of cells in the sub-G1/G0 apoptotic phase (FIG. 2D).

We subsequently analysed activation of the Fas pathway in MCF-7 cells following co-treatment with 5-FU and CH-11. As already noted, treatment with 5-FU alone resulted in dramatic up-regulation of Fas, but had no effect on caspase 8 activation (FIG. 2E). However, co-treatment of MCF-7 cells with 5-FU and CH-11 resulted in a dramatic activation of caspase 8 as indicated by complete loss of procaspase 8 (FIG. 2E). Furthermore, cleavage of PARP (poly(ADP) ribose polymerase), a hallmark of apoptosis, was only observed in MCF-7 cells co-treated with 5-FU and CH-11 (FIG. 2E). We next analysed the kinetics of caspase 8 activation in 5-FU and CH-11 co-treated cells. Caspase 8 was potently activated 12 hours after addition of CH-11 to 5-FU pre-treated cells (FIG. 2F). Importantly, this coincided with complete processing of c-FLIP_{L} to its p43-form (FIG. 2F). By 24 hours after the addition of CH-11, neither procaspase 8 nor c-FLIP_{L} (both its full-length and truncated forms) was detected.

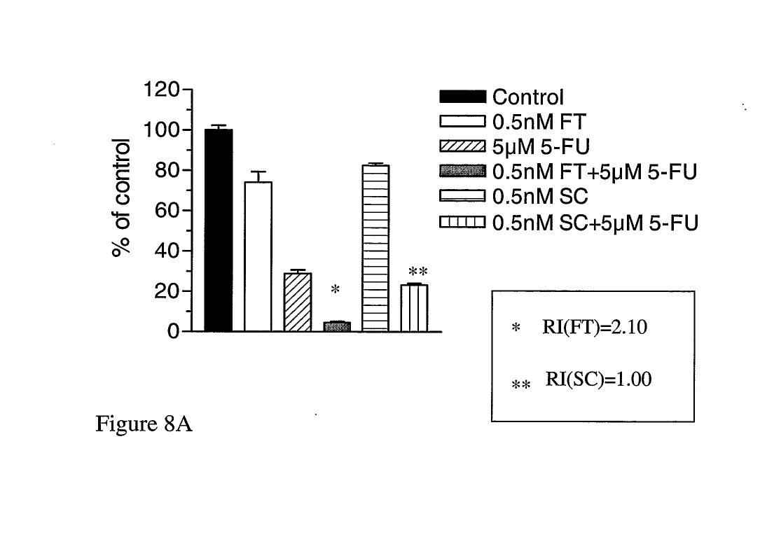

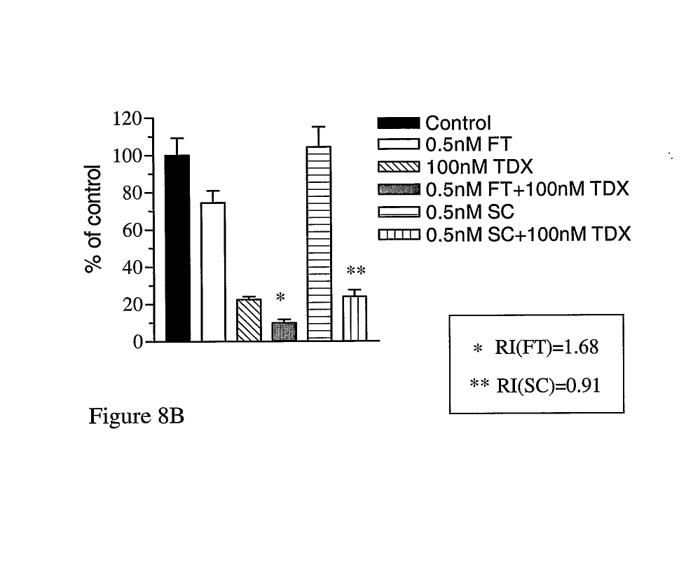

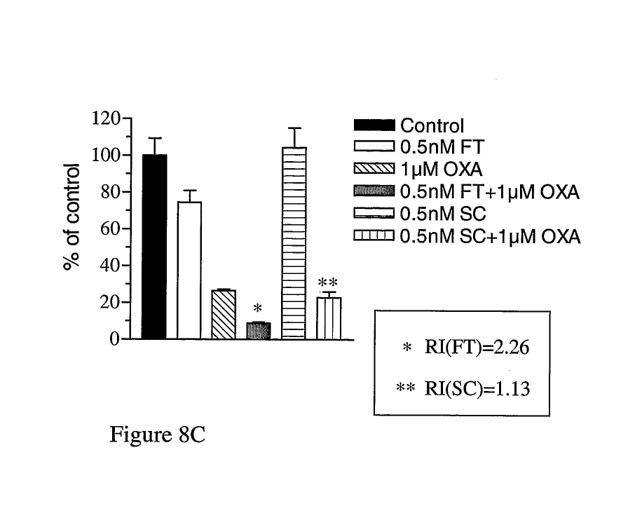

Similarly, treatment of HCT116p53^{+/+} cells with IC₆₀₍₇₂ₕ₎ doses of 5-FU (5 μM) or oxaliplatin (1 μM) for 48 hours resulted in potent up-regulation of Fas expression (FIG. 8A), but only modest activation of caspase 8 and no PARP cleavage (FIG. 8B). However, co-treatment with each drug and CH-11 resulted in potent activation of caspase 8 and PARP cleavage (FIG. 8B). Activation of caspase 8 correlated with the complete processing of c-FLIP_{L} to p43-FLIP_{L} in drug and CH-11 co-treated cells (FIG. 8B). Furthermore, addition of CH-11 to 5-FU- and oxaliplatin-treated HCT116p53^{+/+} cells resulted in ˜4- and ˜8-fold up-regulation of c-FLIP_{S} respectively (FIG. 8B). These results suggested the involvement of c-FLIP in regulating Fas-mediated apoptosis in HCT116p53^{+/+} cells following chemotherapy.

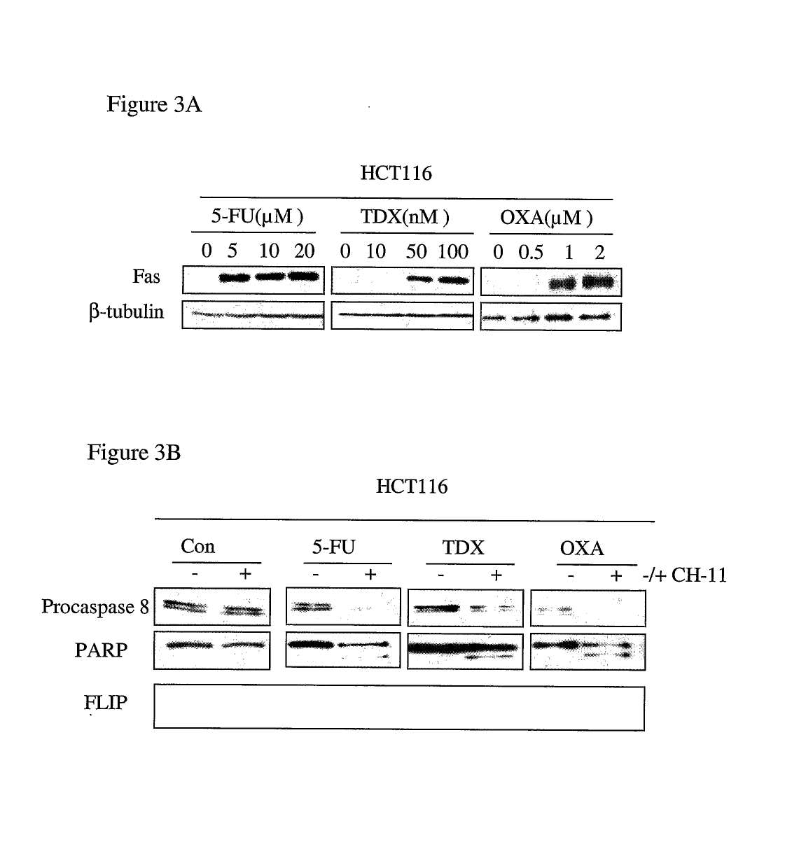

We also examined the ability of CH-11 to activate apoptosis in the HCT116 colon cancer cell line. Fas was potently up-regulated in HCT116 cells 48 hours after treatment with 5-FU, TDX and OXA (FIG. 3A). Treatment with each drug alone or CH-11 alone for 48 hours failed to significantly activate caspase 8 or induce PARP cleavage (FIG. 3B). However, treatment with each drug for 24 hours followed by CH-11 for a further 24 hours resulted in activation of caspase 8 and PARP cleavage. Importantly, activation of caspase 8 correlated with processing of c-FLIP_{L} in drug and CH-11 co-treated cells (FIG. 3B).

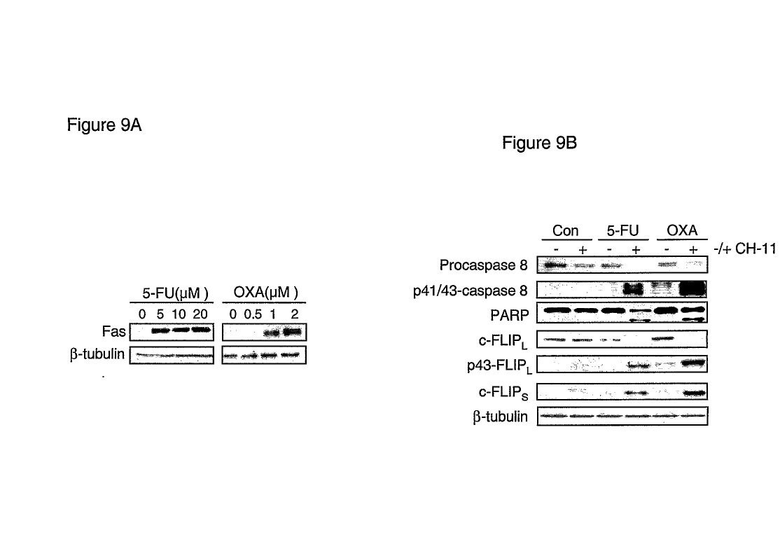

To further test the hypothesis that the intracellular signal to commit to death receptor-mediated apoptosis in HCT116p53^{+/+} cells following drug treatment was regulated by c-FLIP, the inventors generated HCT116p53^{+/+} cell lines that overexpressed c-FLIP_{L} or c-FLIP_{S}. The HFL17 and HFL24 cell lines both overexpressed c-FLIP_{L} by ˜6-fold compared to cells transfected with a LacZ-expressing construct (HLacZ), while the HFS19 and HFS44 cell lines overexpressed c-FLIP_{S} by ˜5- and ˜10-fold respectively compared to the control cell line (FIG. 9A). Growth inhibition studies (MTT assays) were carried out to determine the IC₅₀₍₇₂ₕ₎ dose for each chemotherapy in each cell line. It was found that overexpressing c-FLIP_{S} had no significant effect on the IC₅₀₍₇₂ₕ₎ dose of any of the drugs, while c-FLIP_{L} overexpression caused a moderate 1.7-2.0-fold increase in the IC₅₀₍₇₂ₕ₎ dose of oxaliplatin, but had no effect on the IC₅₀₍₇₂ₕ₎ doses of the other drugs (Table 1).

Flow cytometry revealed that c-FLIP_{L} overexpression did not affect cell cycle arrest in response to chemotherapy, but had a marked effect on chemotherapy-induced apoptosis (FIG. 9B). For example, treatment with 5 μM 5-FU for 72 hours resulted in cell cycle arrest at the G1/S phase boundary in each cell line, however the levels of apoptosis in the two c-FLIP_{L}-overexpressing lines was significantly reduced compared to the control cell line, with ˜15% of HFL17 cells and ˜17% of HFL24 cells in the sub-G₁/G₀ apoptotic fraction compared to ˜41% in the HLacZ cell line (p<0.0001, FIG. 9B). In contrast, the levels of apoptosis induced by 5-FU in the two c-FLIP_{S}-overexpressing lines were actually somewhat higher than in the control HLacZ cell line. Similar results were obtained with the other drugs, as overexpression of c-FLIP_{L} significantly decreased oxaliplatin- and CPT-11-induced apoptosis, whereas c-FLIP_{S} overexpression failed to inhibit chemotherapy-induced apoptosis (FIG. 9B). The similar IC₅₀₍₇₂ₕ₎ doses observed in the c-FLIP_{L}-overexpressing cell lines and the HLacZ cell line (Table 1) probably reflects the fact that c-FLIP_{L} overexpression did not affect chemotherapy-induced cell cycle arrest, resulting in similar levels of growth inhibition despite the differences in drug-induced apoptosis observed in these cell lines.

### Example 4

### Overexpression of c-FLIP_{L} Inhibits Chemotherapy-Induced Fas-Mediated Cell Death

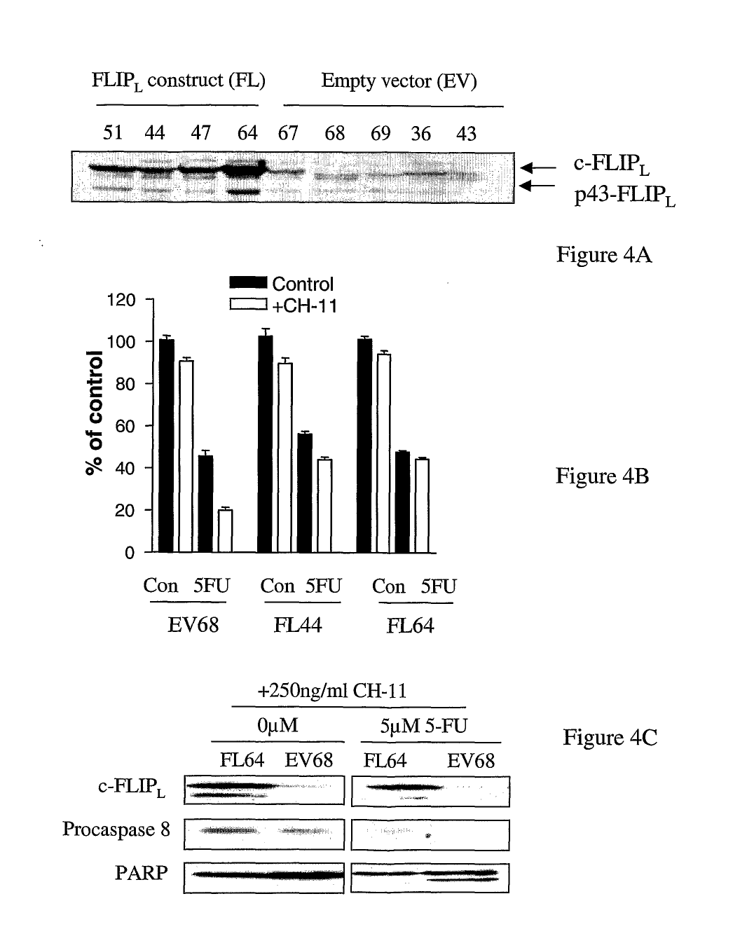

To further investigate the role of c-FLIP_{L} in regulating Fas-mediated apoptosis following drug treatment, we developed a panel of MCF-7 cell lines overexpressing c-FLIP_{L}. We developed cell lines with 5-10-fold increased c-FLIP_{L} expression compared to cells transfected with empty vector (FIG. 4A). The c-FLIP_{L}-overexpressing cell lines FL44 and FL64 and cells transfected with empty vector (EV68) were taken forward for further characterisation. Cell viability assays indicated that treatment of EV68 cells with 5-FU followed by CH-11 resulted in a highly synergistic decrease in cell viability (RI=2.06, p<0.0005) (FIG. 4B). However, no synergistic decrease in cell viability was observed in 5-FU and CH-11 co-treated FL44 or FL64 cells, with RI values of 1.14 and 1.01 respectively (FIG. 4B). Furthermore, 5-FU and CH-11 co-treatment resulted in caspase 8 activation and PARP cleavage in EV68 cells (FIG. 4C). In contrast, c-FLIP_{L} overexpression in FL64 cells abrogated both activation of caspase 8 and PARP cleavage in response to 5-FU and CH-11 co-treatment (FIG. 4C).

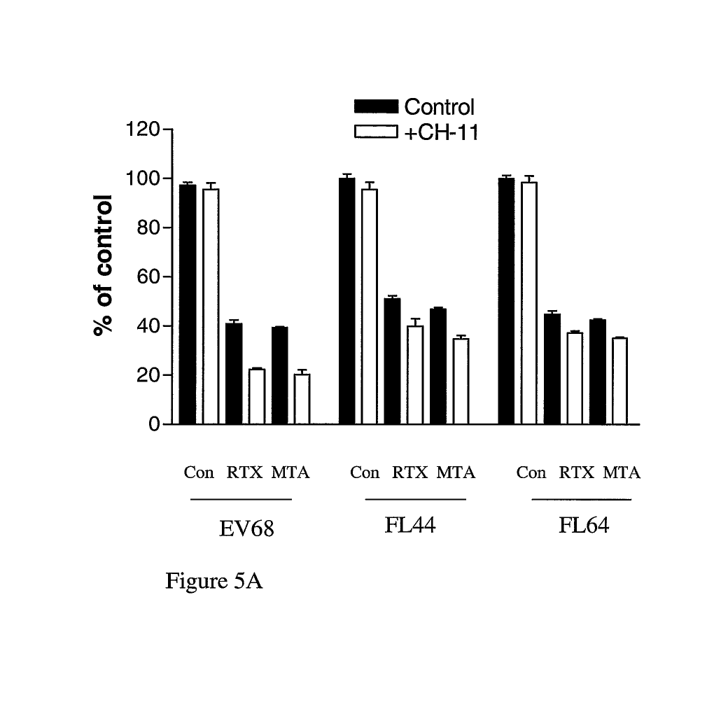

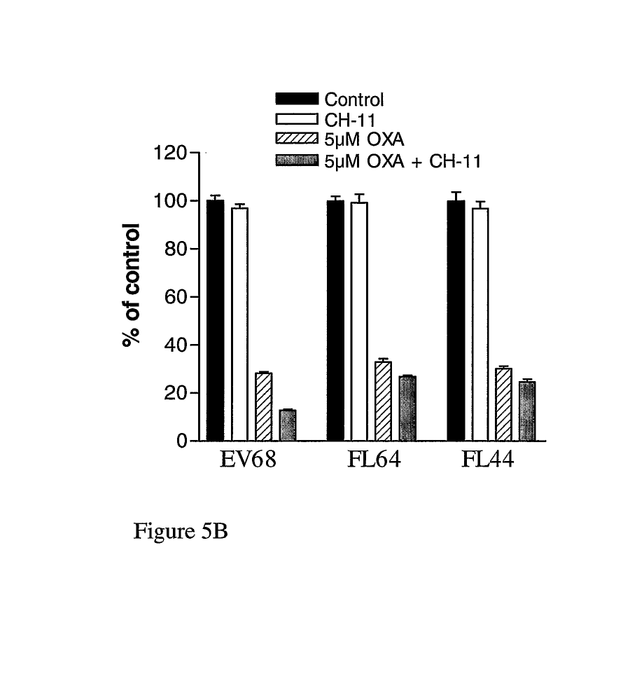

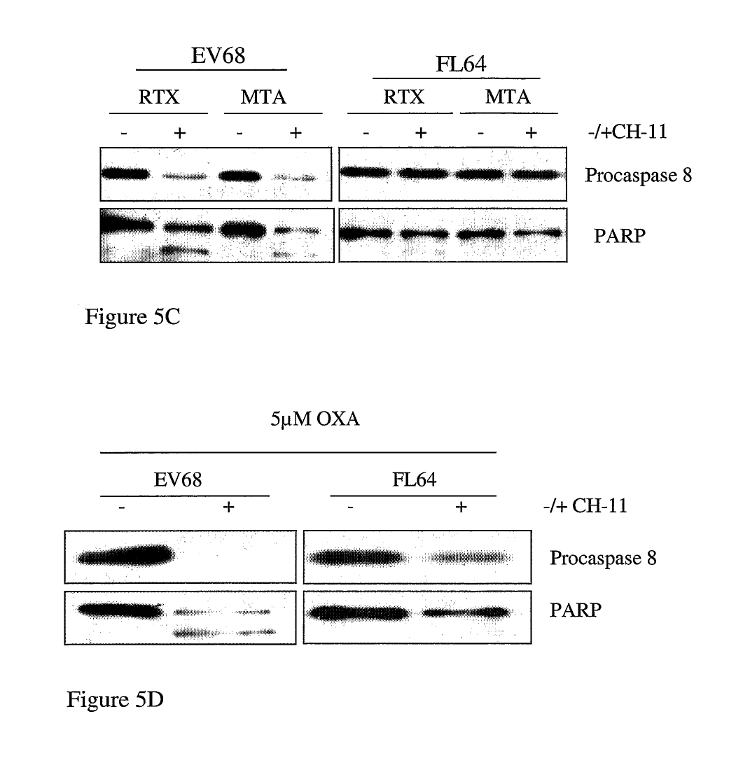

We next examined the effect of c-FLIP_{L} overexpression on Fas-mediated apoptosis following treatment with the antifolates TDX and MTA and the DNA-damaging agent OXA. All three drugs synergistically decreased cell viability in EV68 cells when combined with CH-11 (FIGS. 5A and B). However, this synergistic interaction was inhibited by c-FLIP_{L} overexpression in both the FL44 and FL64 cell lines (FIGS. 5A and B). Analysis of caspase 8 activation and PARP cleavage confirmed that Fas-mediated apoptosis in response to all three agents was attenuated by c-FLIP_{L} overexpression. Combined treatment with each antifolate and CH-11 resulted in caspase 8 activation in EV68 cells, but not FL64 cells (FIG. 5C). Similarly, PARP cleavage in response to the antifolates and CH-11 was inhibited in the FL64 cell line (FIG. 5C). Although some caspase 8 activation and PARP cleavage were observed in FL64 cells following co-treatment with 5 μM OXA and CH-11, this was much reduced compared to the EV68 cell line (FIG. 5D). These results indicate that c-FLIP_{L} is a key regulator of Fas-mediated apoptosis in response to 5-FU, antifolates and oxaliplatin.

Similar experiments were carried out using a number of other cell lines and chemotherapeutic agents in combination with CH-11. The results are shown in FIG. 9C. Treatment with 50 ng/mL CH-11 in the absence of chemotherapy induced a small degree of apoptosis in the HLacZ control cell line (data not shown). However, co-treatment with each chemotherapy and CH-11 resulted in high levels of apoptosis in the HLacZ cell line (FIG. 9C). High levels of apoptosis were also observed in the c-FLIP_{S}-overexpressing cell lines HFS19 and HFS44 in response to chemotherapy and CH-11 (FIG. 9C). In contrast, c-FLIP_{S} overexpression in the HFL17 and HFL24 cell lines dramatically inhibited apoptosis in response to co-treatment with each chemotherapy and CH-11 (FIG. 9C). So, overexpression of c-FLIP_{L}, but not c-FLIP_{S}, protected HCT116p53^{+/+} cells from both chemotherapy-induced apoptosis and apoptosis induced in response to co-treatment with chemotherapy and the Fas agonist CH-11.

### Example 6

### siRNA-Targeting of c-FLIP Sensitises Cancer Cells to Chemotherapy

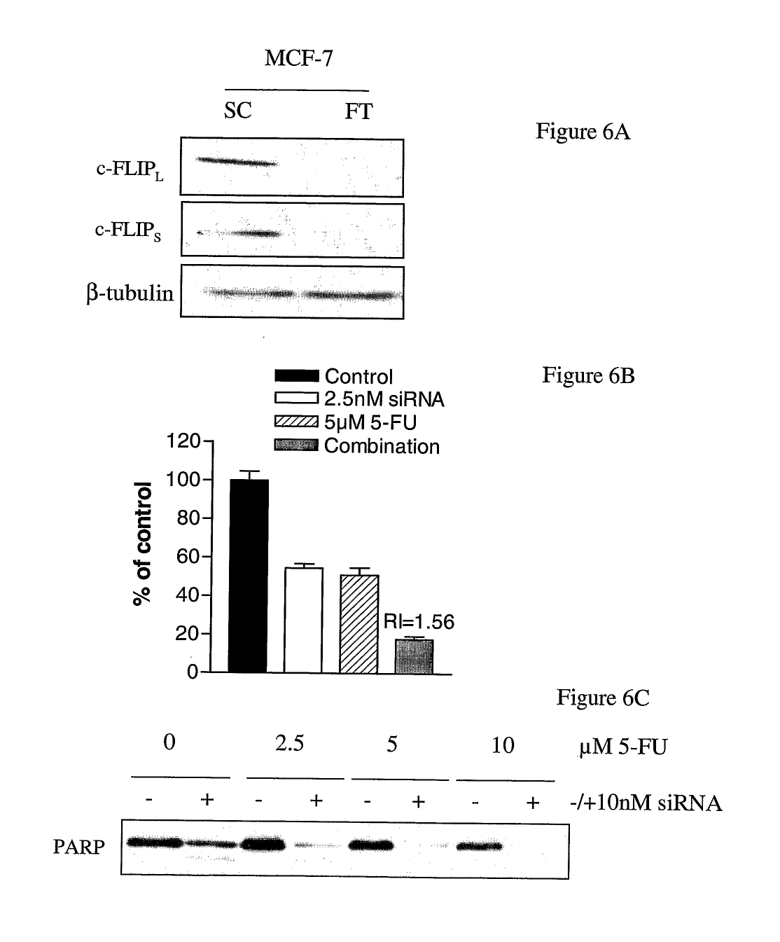

Having established that c-FLIP_{L} overexpression protected MCF-7 and HCT116 cells from chemotherapy-induced Fas-mediated cell death, we next designed a FLIP-targeted (FT) siRNA to inhibit both c-FLIP splice variants. Transfection with 10 nM FT siRNA potently down-regulated expression of both c-FLIP splice variants in MCF-7 cells (FIG. 6A). Cell viability analysis of MCF-7 cells transfected with FT siRNA indicated that co-treatment with 5-FU resulted in a supra-additive decrease in cell viability (FIG. 6B, RI=1.56, p<0.005). Interestingly, transfection of MCF-7 cells with FT siRNA significantly decreased cell viability in the absence of co-treatment with 5-FU, with an approximate 50% decrease in cell viability in cells transfected with 2.5 nM FT siRNA (FIG. 6B). A scrambled control (SC) siRNA that had no effect of FLIP expression, also had no effect on cell viability either alone or in combination with 5-FU (data not shown). The decrease in cell viability in response to FT siRNA alone appeared to be due to the induction of apoptosis, as transfection of FT siRNA in the absence of co-treatment with drug induced significant levels of PARP cleavage (FIG. 6C, lane 2). Furthermore, combined treatment with FT siRNA and 5-FU resulted in potent cleavage of PARP (FIG. 6C), indicating that the synergistic decrease in cell viability observed in MCF-7 cells co-treated with these agents was due to increased apoptosis.

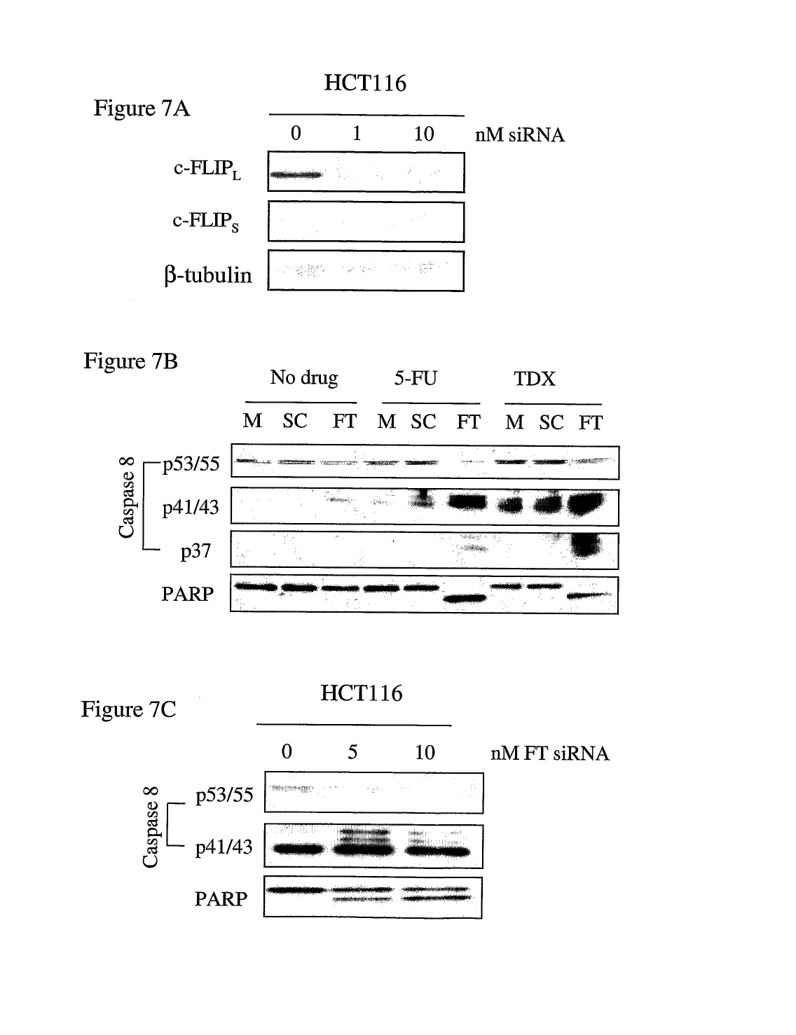

FT siRNA also potently down-regulated FLIP_{L} and FLIP_{S} expression in HCT116 cells (FIG. 7A). Analysis of caspase 8 activation in siRNA-transfected HCT116 cells indicated that FT siRNA alone (1 nM) caused some activation of caspase 8, as indicated by the decrease in the levels of p53/55 zymogen and appearance of the p41/43 cleavage products (FIG. 7B, lane 3). This was accompanied by some PARP cleavage. At higher concentrations (>5 nM), FT siRNA alone caused more potent activation of caspase 8 and PARP cleavage in HCT116 cells (FIG. 7C). Both 5-FU (5 μM) and TDX (100 nM) caused some caspase 8 activation in mock and SC transfected HCT116 cells as indicated by the presence of p41/p43 caspase 8, although no PARP cleavage was observed in these cells (FIG. 7B). The most potent activation of caspase 8 was observed in cells co-treated with 1 nM FT siRNA and 5-FU or TDX, with decreased expression of the p53/55 zymogen and increased expression of both the p41/43 and p18 caspase 8 cleavage products (FIG. 7B, lanes 6 and 9). Furthermore, activation of caspase 8 in FT siRNA/chemotherapy-treated HCT116 cells was accompanied by potent PARP cleavage. Cell viability assays indicated that co-treatment with 0.5 nM FT siRNA and 5 μM 5-FU resulted in a synergistic decrease in cell viability (FIG. 8A, RI=2.10, p<0.0005). In contrast, SC siRNA had no significant effect on cell viability either in the presence or absence of 5-FU. Furthermore, co-treatment with FT siRNA and both TDX and OXA resulted in synergistic decreases in cell viability, with RI values of 1.68 and 2.26 respectively (FIGS. 8B and C). These results indicate that inhibition of c-FLIP expression in HCT116 and MCF-7 cells dramatically sensitised them to chemotherapy-induced apoptosis.

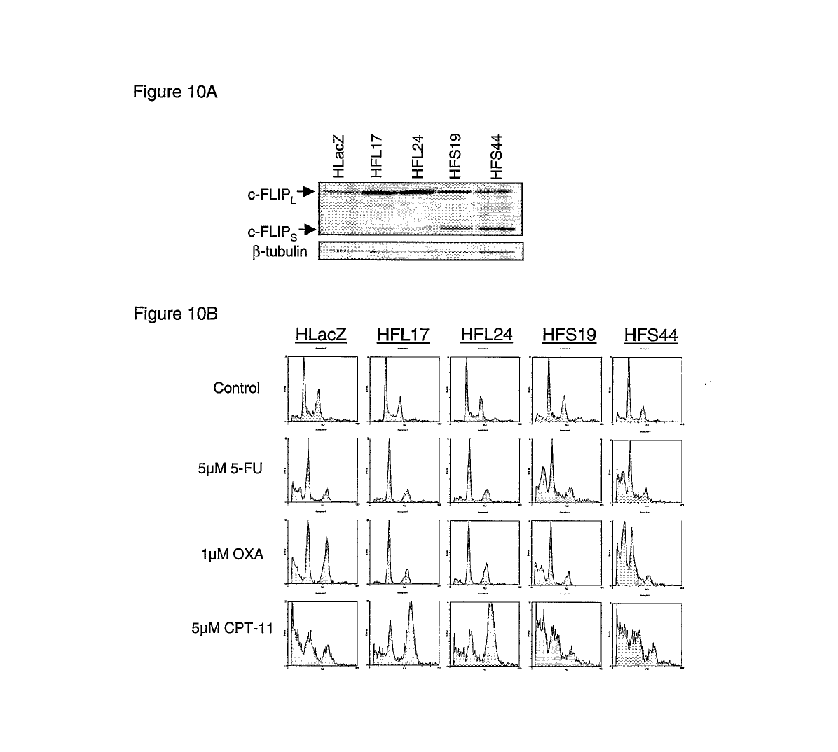

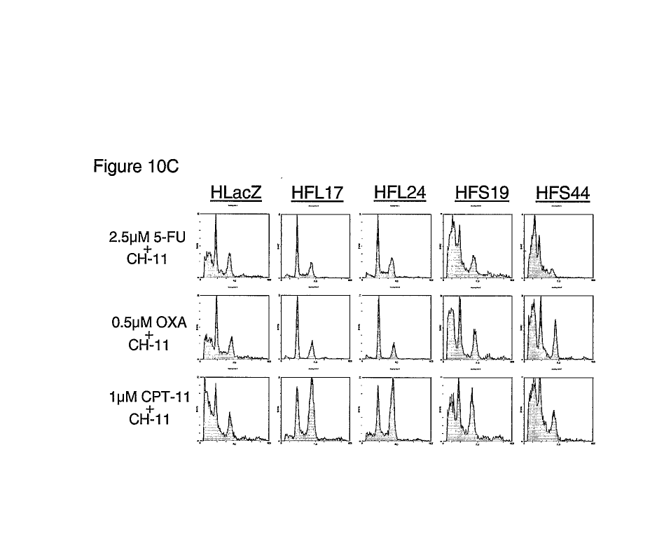

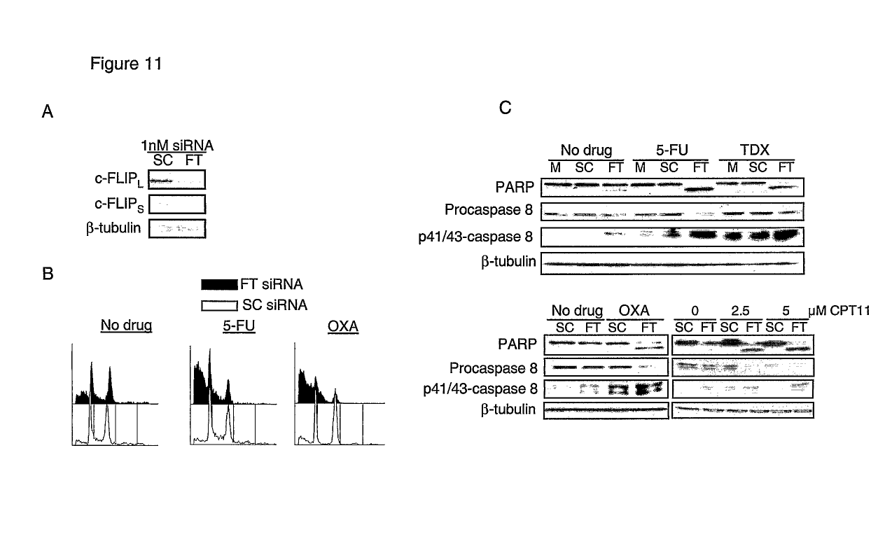

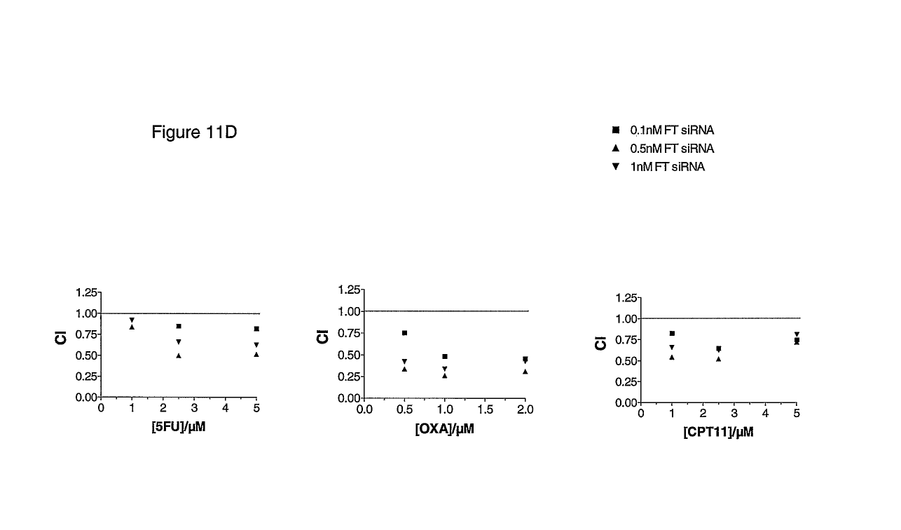

Further evidence that siRNA-targeting of c-FLIP sensitises HCT116p53^{+/+} cells to chemotherapy is shown in FIG. 11. FLIP-targeted siRNAs were designed to down-regulate expression of both c-FLIP splice variants. Of several siRNAs tested, one FLIP-targeted (FT) siRNA potently down-regulated expression of both c-FLIP splice variants in HCT116p53^{+/+} cells at nanomolar concentrations (FIG. 11A). We used this FT siRNA to analyse the effect of down-regulating c-FLIP expression on drug-induced apoptosis. Interestingly, transfection with 0.5 nM FT siRNA in the absence of chemotherapy induced significant levels of apoptosis (˜26%) in HCT116p53^{+/+} cells compared to cells transfected with control siRNA (˜9%) as assessed by flow cytometric analysis of cells in the sub-G₀/G₁ apoptotic fraction (p<0.0001; FIG. 11B). Importantly, co-treatment of FT siRNA transfected cells with an IC60₇₂ₕ dose of 5-FU for 48 hours resulted in a supra-additive increase in apoptosis, with ˜43% of cells undergoing apoptosis compared to ˜11% in 5-FU-treated cells transfected with the control non-silencing siRNA (p=0.0018; FIG. 11B). The results following oxaliplatin treatment were even more dramatic, with ˜61% of cells co-treated with FT siRNA and oxaliplatin in the sub-G₁/G₀ phase after 48 hours, compared to ˜17% of cells co-treated with control siRNA and oxaliplatin (p<0.0001; FIG. 11B). Analysis of caspase 8 activation in siRNA-transfected HCT116p53^{+/+} cells indicated that 0.5 nM FT siRNA alone caused some activation of caspase 8, as indicated by the decrease in the levels of p53/55 zymogen and appearance of the p41/43 cleavage products (FIG. 1C). Consistent with the cell cycle data, transfection with 0.5 nM FT siRNA resulted in some PARP cleavage in the absence of chemotherapy. Treatment with 5 μM 5-FU also caused modest caspase 8 activation in mock-transfected cells and cells transfected with control siRNA (as indicated by the presence of p41/p43 caspase 8), however no PARP cleavage was observed in these cells (FIG. 11C). By far the most potent activation of caspase 8 was observed in cells co-treated with 0.5 nM FT siRNA and 5-FU, with decreased expression of the p53/55 zymogen and increased expression of the p41/43 caspase 8-cleavage product (FIG. 1C). Furthermore, activation of caspase 8 in FT siRNA/5-FU-treated HCT116p53^{+/+} cells was accompanied by complete PARP cleavage. Similar results were obtained for the antifolate tomudex, which is a specific inhibitor of nucleotide synthetic enzyme thymidylate synthase (TS) (FIG. 11C). Furthermore, potent caspase 8 activation and PARP cleavage were observed in cells co-treated with FT siRNA and oxaliplatin after 24 hours, compared to cells treated with either agent individually (FIG. 11C). In light of these results, we also examined the effect of down-regulating c-FLIP on apoptosis induced by CPT-11, another chemotherapeutic drug currently used in the treatment of colorectal cancer. As with the other drugs, down-regulation of c-FLIP sensitised HCT116p53^{+/+} cells to CPT-11-induced activation of caspase 8 and apoptosis (FIG. 10C).

Given the more than additive effects of FT siRNA and chemotherapy on apoptosis in HCT116p53^{+/+} cells, we carried out cell viability assays to determine whether the interactions were synergistic. Cell viability assays indicated that co-treatment with FT siRNA and 5-FU resulted in combination index (CI) values of <1 for 8/9 concentrations (FIG. 11D). According to the definitions of Chou and Talalay, the CI values for FT siRNA/5-FU co-treatment indicated that there was a moderate synergistic interaction for 4/9 concentration combinations examined and a synergistic interaction for a further 4 concentrations (FIG. 11D). Co-treatment with FT siRNA and oxaliplatin resulted in synergistic decreases in cell viability for all concentrations examined, with CI values ranging from ˜0.25-0.75 (FIG. 3D). Similarly, combined treatment with CPT-11 and FT siRNA resulted in synergistic or moderate synergistic decreases in cell viability with CI values ranging from ˜0.50-0.85 (FIG. 11D). Control siRNA had no effect on cell viability in the presence or absence of any of the drugs (data not shown). Collectively, these results indicate that down-regulation of c-FLIP expression dramatically sensitises HCT116p53^{+/+} cells to 5-FU-, oxaliplatin- and CPT-11-induced apoptosis.

### Example 7A

### The Agonistic Fas Monoclonal Antibody CH-11 Synergistically Activates Apoptosis in Response to CPT-11 and TDX in a p53-Independent Manner

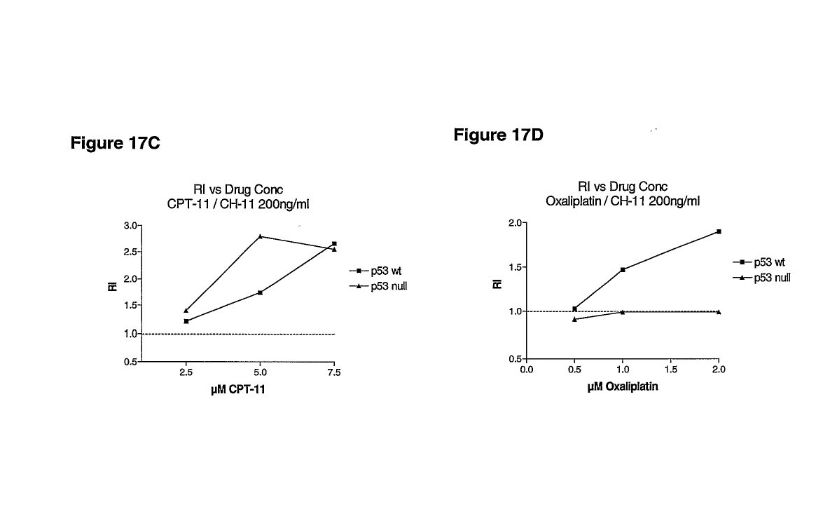

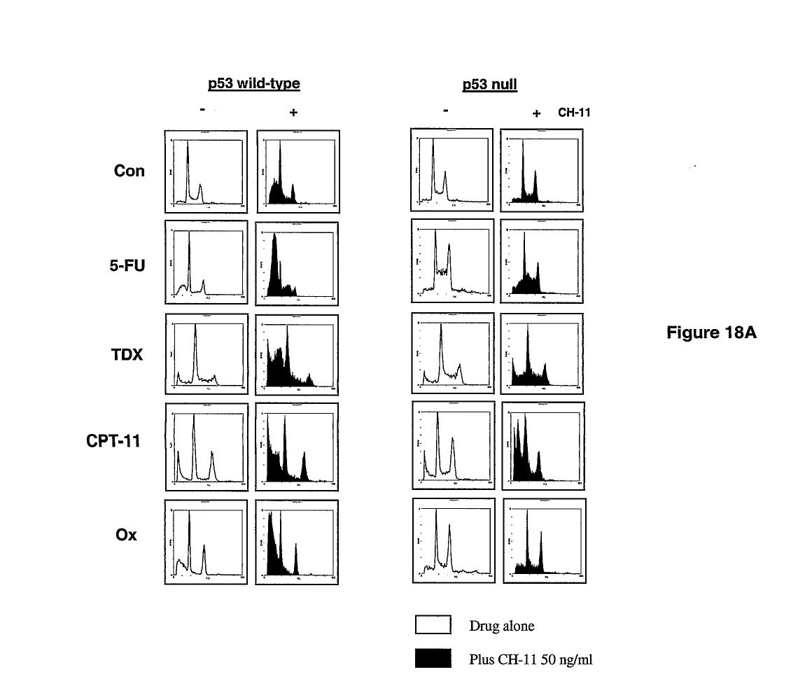

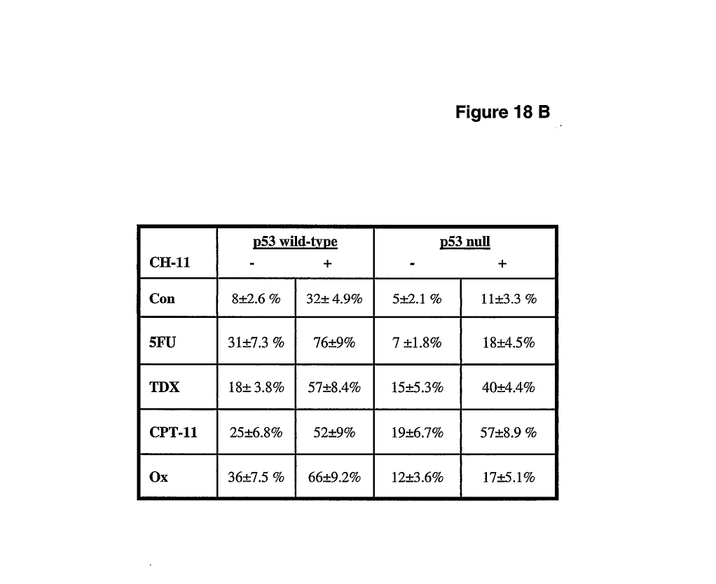

The agonistic anti-Fas antibody CH-11 has been shown to activate the Fas/CD95 receptor and cause apoptosis. Lack of up-regulation of the Fas/CD95 receptor in a p53 mutant colon cancer cell line abolished the synergistic interaction between 5-FU and CH-11. In our study treatment of the p53 wild-type and null cell lines with a range of each of the chemotherapy agents 5-FU, TDX, CPT-11 and Oxaliplatin followed 24 hours later by the addition of the anti-Fas antibody CH-11 (200 ng/ml) for a further 48 hours resulted in significant synergy for all the drugs in the p53 wild-type setting, but in the p53 null cells this synergy was only seen with the topoisomerase-I inhibitor CPT-11 and the thymidylate synthase inhibitor TDX. There was no synergistic interaction seen at all with Oxaliplatin in the p53 null cells at any dose, and only slight interaction with 5-FU at the higher doses (FIG. 17). Propidium iodide staining of the HCT116 p53 wild-type and null cell lines treated with these chemotherapeutic agents for 24 hours followed by CH-11 50 ng/ml for an additional 24 hours confirmed that a synergistic interaction is seen with each of the drugs and CH-11 in the p53 wild-type cells (FIG. 18), whereas in the p53 null setting only treatment with CPT-11 and to a lesser extent with TDX resulted in significant synergy with CH-11 50 ng/ml.

### Example 7B

### Effect of p53 Inactivation on the Synergy Between CH-11 and 5-FU, CPT-11 and Oxaliplatin

Activation of the Fas/CD95 receptor by its natural ligand FasL or the monoclonal antibody CH-11 results in the recruitment and activation of procaspase 8 at the DISC. Procaspase 8 is cleaved to its active subunits p41/43 and p18. Poly(ADP-ribose)polymerase (PARP) is normally involved in DNA repair and stability, and is cleaved by members of the caspase family during early apoptosis.

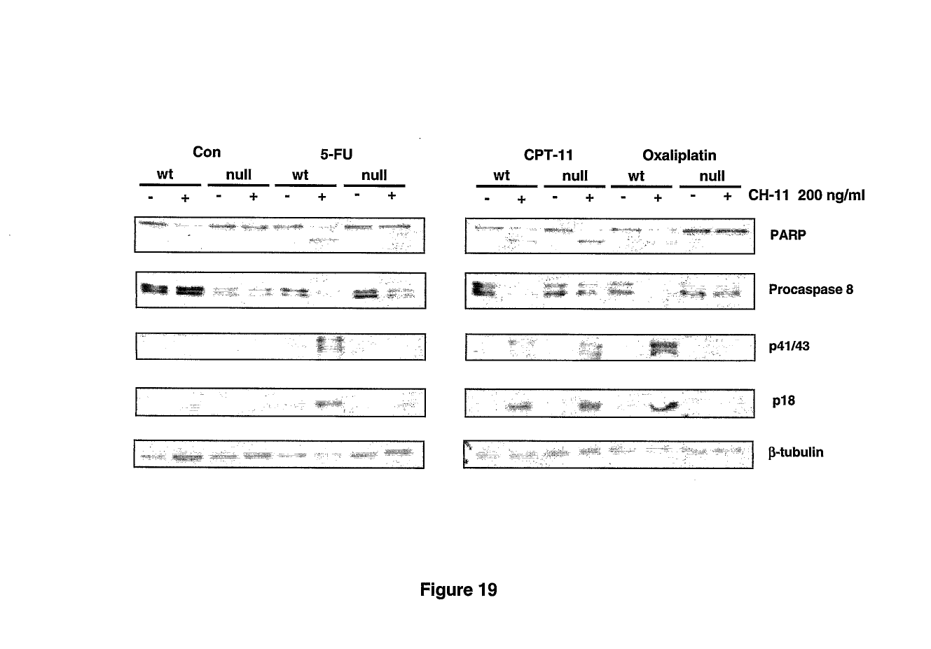

Western blot analysis of the p53 wild-type and null cell lines treated with IC60 doses of these chemotherapeutic agents for 24 hours followed by a further 24 hours of the anti-Fas antibody CH-11 (200 ng/ml) resulted in PARP cleavage and activation of procaspase 8 (with generation of the active p41/43 and p18 subunits) in the p53 wild-type cell line for each drug (FIG. 19). In the p53 null cell line PARP cleavage and procaspase 8 activation following the addition of CH-11 was only seen following treatment with CPT-11.

### Example 7C

### Effect of p53 Status on c-FLIP Regulated Chemosensitivity

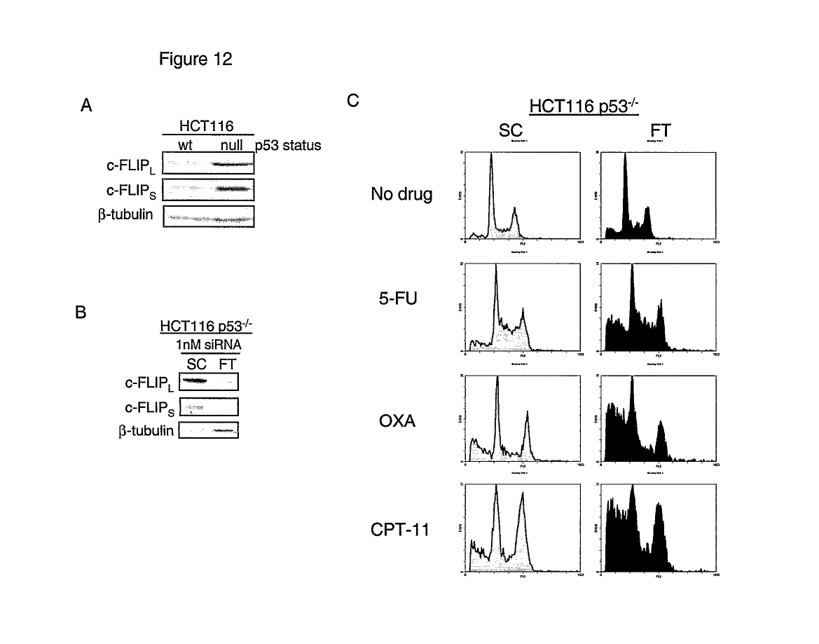

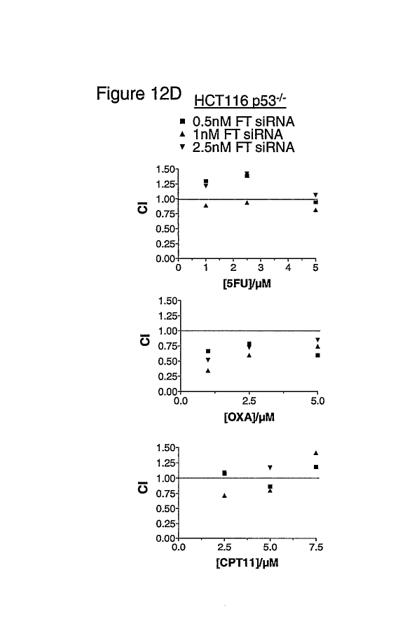

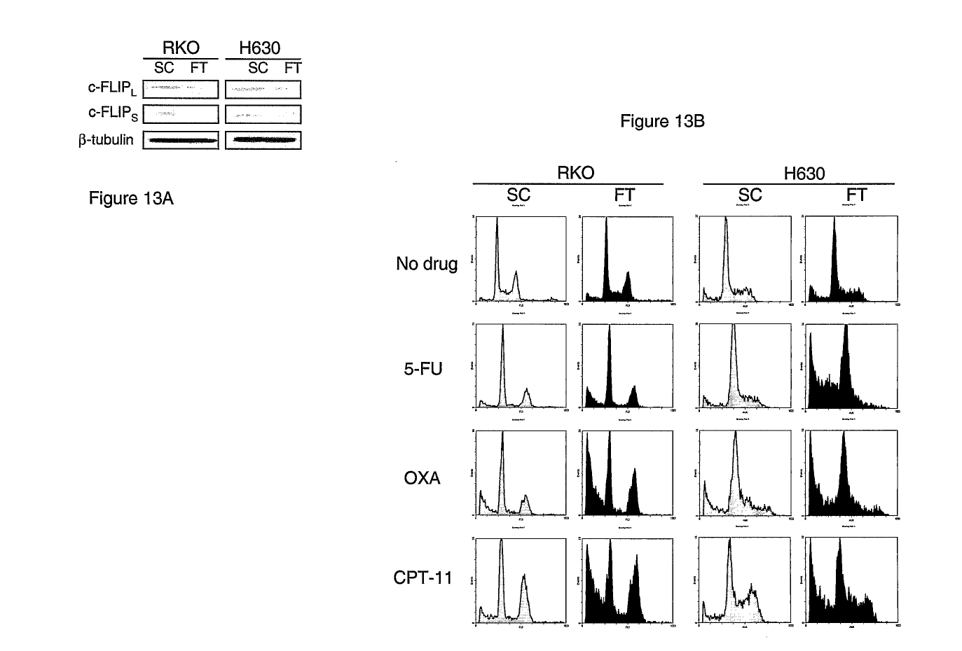

In order to determine whether down-regulation of c-FLIP would also sensitise p53 null HCT116 cells to chemotherapy-induced apoptosis, we transfected these cells with FT siRNA and co-treated them with chemotherapy (5-FU, oxaliplatin and CPT-11). The p53 null cells (HCT116p53−/−) expressed higher levels of both c-FLIP splice forms than p53 wild type cells (FIG. 12A), but expression was effectively down-regulated by 1 nM FT siRNA (FIG. 12B). Treatment of the p53 null cells with 1 nM FT siRNA alone resulted in a modest increase in apoptosis after 72 hours, with ˜14% of cells in the sub-G₀/G₁ fraction compared to ˜9% in SC siRNA transfected cells (p=0.0081; FIG. 12C). Co-treatment of FT siRNA-transfected cells with 5 μM 5-FU significantly increased the apoptotic fraction to ˜29% compared to ˜14% of 5-FU/SC siRNA co-treated cells (p=0.0003; FIG. 12C). Treatment of FT siRNA-transfected HCT116 p53 null cells with 5 μM oxaliplatin resulted in a highly significant increase in cells undergoing apoptosis compared to oxaliplatin/SC siRNA co-treated cells (−46% compared to ˜27%, p<0.0001; FIG. 4C). FT siRNA also increased apoptosis of HCT116p53^{-/-} cells in response to 1 μM CPT-11 to ˜33% compared to ˜22% in SC/CPT-11 co-treated cells (p=0.0002; FIG. 12C). These results indicate that down-regulating c-FLIP expression significantly enhanced chemotherapy-induced apoptosis in p53 null HCT116 cells, in particular oxaliplatin-induced apoptosis.

We further analysed the effect of down-regulating c-FLIP on the chemosensitivity of p53 null HCT116 cells using the MTT cell viability assay. While greater than additive increases in apoptosis were detected for combined treatment with FT siRNA and 5-FU in HCT116p53^{-/-} cells (FIG. 12C), cell viability assays identified slight synergy in only 2/9 combinations (FIG. 12D). Similarly, the interaction between FT siRNA and CPT-11 was found to be moderately or slightly synergistic for only 3/9 drug combinations (FIG. 12D). So, although c-FLIP down-regulation sensitised HCT116p53−/− cells to 5-FU- and CPT-11-induced apoptosis (FIG. 12C), cell viability assays indicated that fewer drug combinations were synergistic than in the p53 wild type parental cell line, and that the degree of synergy was less. However, co-treatment of HCT116p53−/− cells with oxaliplatin and FT siRNA was synergistic or moderately synergistic for all nine combinations analysed, with CI values ranging from ˜0.35-0.85 (FIG. 12D), most likely reflecting the greater level of apoptosis induced for this combination than for the other chemotherapeutic drugs (FIG. 12C).

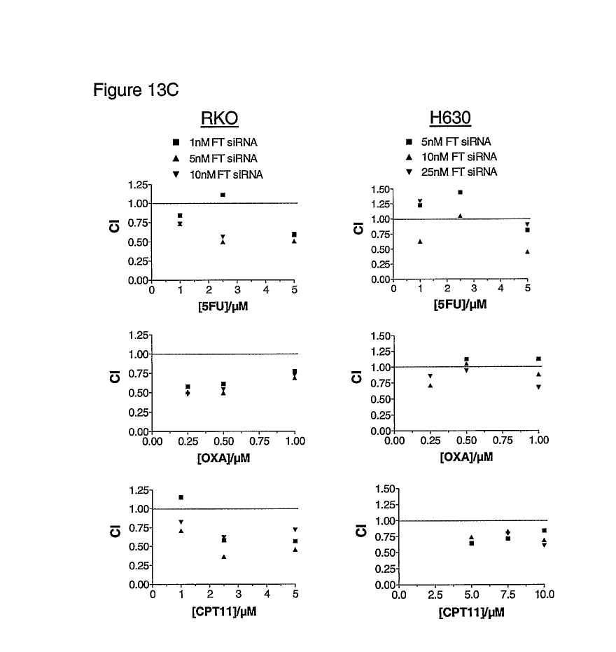

Effect of c-FLIP on chemosensitivity in other colorectal cancer cell lines. In order to determine whether c-FLIP is a general modulator of chemosensitivity in colorectal cancer, we extended these studies into two further colorectal cancer cell line models, namely the p53 wild type RKO cell line and the p53 mutant H630 cell line. Each cell line expressed both c-FLIP splice forms, and FT siRNA down-regulated c-FLIP protein in both lines (FIG. 13A). As in the HCT116 cell lines, down-regulation of c-FLIP sensitised both cell lines to apoptosis induced by 5-FU, oxaliplatin and CPT-11 (FIG. 5B). In each case, the effect of co-treatment with chemotherapy and FT siRNA was more than additive. Of note, the sensitisation to CPT-11 was particularly marked in both lines, with ˜43% of FT siRNA/CPT-11 co-treated RKO cells undergoing apoptosis compared to ˜15% of SC siRNA/CPT-11 co-treated RKO cells, and ˜32% of FT siRNA/CPT-11 co-treated H630 cells undergoing apoptosis compared to

−12% of SC siRNA/CPT-11 co-treated H630 cells. MTT analyses indicated synergistic interactions between FT siRNA and each drug in RKO cells, with the majority of CI values below 0.75 for each drug (FIG. 13C). The synergy was less pronounced in the H630 cells, with the combination of FT siRNA and CPT-11 being the most consistently synergistic or moderately synergistic (FIG. 13C).

Collectively, these results indicate that c-FLIP plays an important role in regulating chemotherapy-induced apoptosis in colorectal cancer cell lines. Furthermore, while both p53 wild type, mutant and null cell lines are sensitised to chemotherapy-induced apoptosis following down-regulation of c-FLIP, the extent of synergy would appear to be less in cell lines lacking functional p53.

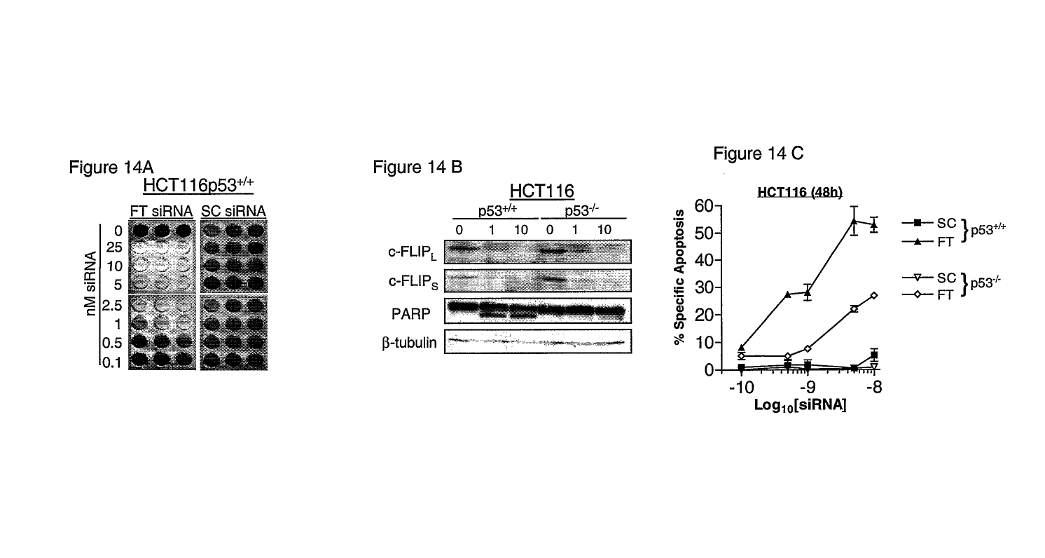

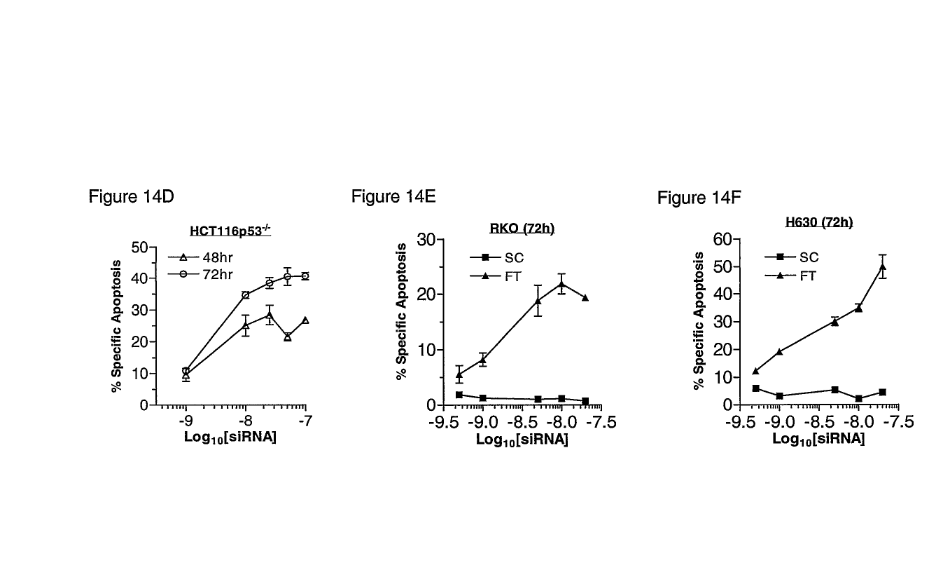

Potent knock-down of c-FLIP induces apoptosis in the absence of chemotherapy. As already discussed, transfection of 0.5 nM FT siRNA into HCT116p53^{+/+} cells significantly increased apoptosis in the absence of co-treatment with chemotherapy (FIG. 10B). When higher concentrations of FT siRNA were used to more completely knock down expression of c-FLIP in HCT116p53^{+/+} cells, a dramatic decrease in cell viability (FIG. 14A) and a significant increase in PARP cleavage and apoptosis was observed (FIGS. 14B and C) in the absence of chemotherapy. A similar effect was observed in HCT116p53^{-/-} cells, although the extent of PARP cleavage and apoptosis was less than in the p53 wild type cell line (FIGS. 14B and C). However, exposure of HCT116p53^{-/-} cells to higher concentrations of FT siRNA for 72 hours resulted in levels of apoptosis that approached those observed in the p53 wild type parental cell line (FIG. 14D). The IC₅₀₍₇₂ₕ₎ doses of FT siRNA in the p53 wild type and null cell lines were ˜0.7 nM and ˜2.5 nM respectively as determined by MTT assay. FT siRNA also potently induced apoptosis in RKO and H630 cells in the absence of chemotherapy (FIGS. 14E and F). The IC₅₀₍₇₂ₕ₎ doses in these cell lines were calculated to be ˜5 nM in RKO cells and ˜25 nM in H630 cells. These results indicate that c-FLIP may be a general determinant of colorectal cancer cell viability even in the absence of cytotoxic drugs. Furthermore, targeting c-FLIP induced apoptosis in p53 wild type, mutant and null and colorectal cancer cells, suggesting that it may represent an important new therapeutic target for treating this disease.

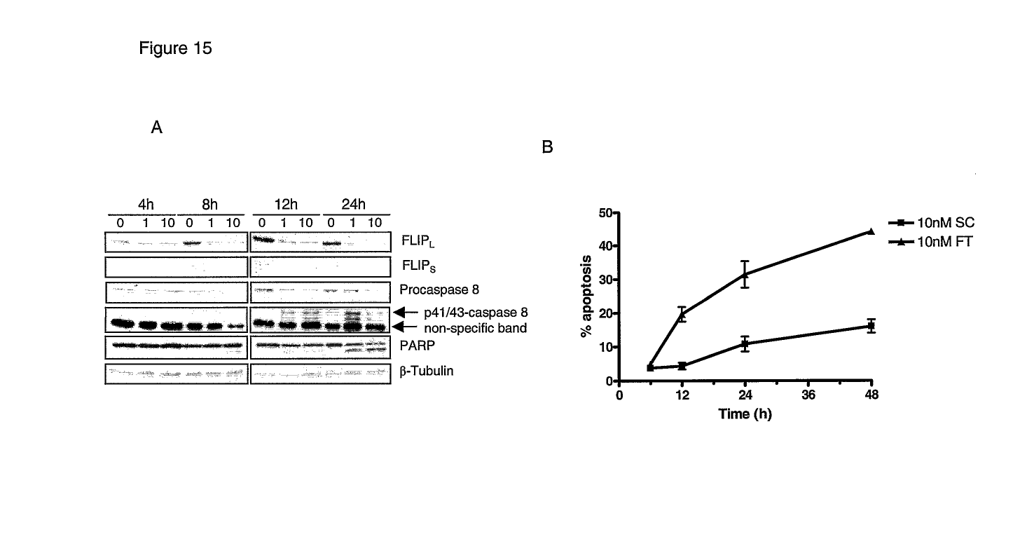

Examination of the kinetics of c-FLIP down-regulation following FT siRNA transfection indicated that both splice forms were efficiently down-regulated as early as 8 hours post-transfection (FIG. 15A). This is in agreement with previous findings, which indicate that c-FLIP is rapidly turned over in cells following treatment with the protein synthesis inhibitor cycloheximide (16). Down-regulation of c-FLIP at 8 hours correlated with decreased levels of procaspase 8 and the onset of apoptosis as indicated by PARP cleavage (FIG. 15A). This was more apparent for the higher concentration of FT siRNA (10 nM). By 12 and 24 hours post-transfection, the p41/43-caspase 8 cleavage fragments could be detected in addition to the decrease in procaspase 8 levels and PARP cleavage in response to 1 nM and 10 nM FT siRNA (FIG. 15A). In agreement with the Western blot analysis, flow cytometry indicated that the onset of apoptosis following FT siRNA transfection occurred between 6 and 12 hours (FIG. 15B). Therefore, c-FLIP down-regulation would appear to be tightly coupled to caspase 8 activation and the onset of apoptosis.

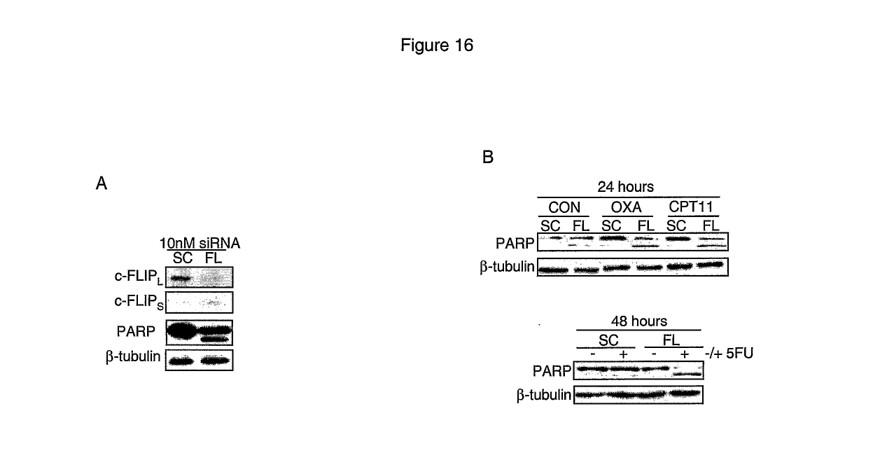

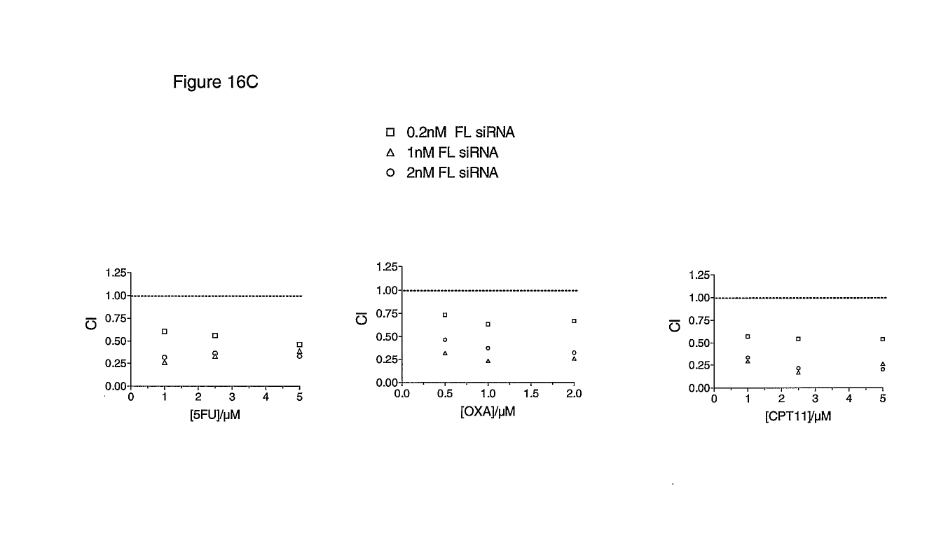

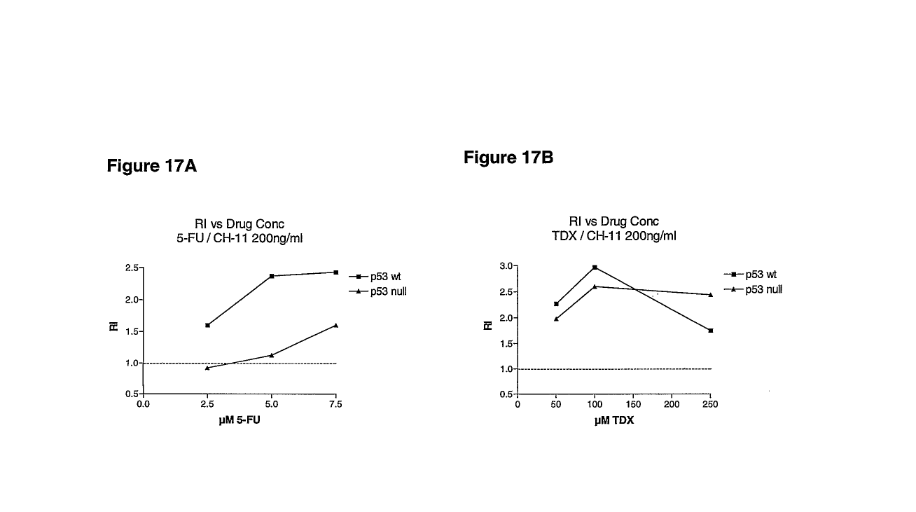

Effect of specific targeting of c-FLIP_{L} on apoptosis. Our initial observation was that activation of apoptosis in chemotherapy/CH-11-treated HCT116p53^{+/+} cells coincided with loss of full-length c-FLIP_{L} (FIG. 9B). It was therefore possible that the effects on cell survival of down-regulating both c-FLIP splice variants were actually a result of the down-regulation of c-FLIP_{L}. In addition, data from the c-FLIP overexpressing cell lines suggested that c-FLIP_{L} was the more important regulator of chemoresistance (FIG. 10B). So, we designed an siRNA to specifically down-regulate the long splice form without affecting expression of c-FLIP_{S} (FIG. 16A). Similar to the effect of the dual-targeted siRNA, specific down-regulation of c-FLIP_{L} induced apoptosis of HCT116p53^{+/+} cells in the absence of chemotherapy, as indicated by PARP cleavage (FIG. 8A) and flow cytometry (data not shown). Furthermore, combined treatment with FL siRNA and each chemotherapy resulted in enhanced apoptosis (FIG. 16B) and highly synergistic decreases in cell viability (FIG. 16C). Similar synergistic decreases in cell viability were observed in the H630 and RKO cell lines (data not shown). These data suggest that down-regulation of c-FLIP_{L} is sufficient to recapitulate the effects of down-regulating both splice variants and that, of the two splice forms, c-FLIP_{L} may be the more critical regulator of colorectal cancer cell death.

### Discussion

We found that the Fas death receptor was highly up-regulated in response to 5-FU, the TS-targeted antifolates TDX and MTA and the DNA-damaging agent OXA in MCF-7 breast cancer and HCT116 colon cancer cells, however, this did not result in significant activation of apoptosis. Expression of FasL by activated T cells and natural killer cells induces apoptosis of Fas expressing target cells in vivo (O'Connell et al., 1999). To mimic the effects of these immune effector cells in our in vitro model, we used the agonistic Fas monoclonal antibody CH-11. We found that CH-11 potently activated apoptosis of chemotherapy-treated cells, suggesting that the Fas signalling pathway is an important mediator of apoptosis in response to these agents in vivo. Many tumour cells overexpress FasL, and it has been postulated that tumour FasL induces apoptosis of Fas-sensitive immune effector cells, thereby inhibiting the antitumor immune response (O'Connell et al., 1999). This hypothesis has been supported by both in vitro and in vivo studies (Bennett et al., 1998; O'Connell et al., 1997). The strategy of overexpressing FasL requires that the tumour cells develop resistance to Fas-mediated apoptosis to prevent autocrine and paracrine induction of tumour cell death. The lack of caspase 8 activation that we observed in response to chemotherapy suggests that Fas-mediated apoptosis may be inhibited in MCF-7 and HCT116 and cancer cells, but that co-treatment with CH-11 was sufficient to overcome this resistance and activate Fas-mediated apoptosis.

Fas signalling may be inhibited by c-FLIP, which can inhibit caspase 8 recruitment to and activation at the Fas DISC (Krueger-et al., 2001). Multiple c-FLIP splice variants have been reported, however, only two forms (c-FLIP_{L} and c-FLIP_{S}) have been detected at the protein level (Scaffidi et al., 1999). Both splice variants have death effector domains (DEDs), with which they bind to FADD, blocking access of procaspase 8 molecules to the DISC. c-FLIP_{L} is processed at the DISC as it is a natural substrate for caspase 8, which cleaves it to generate a truncated form of approximately 43 kDa (p43-FLIP_{L}) (Niikura et al., 2002). Cleaved p43-c-FLIP_{L} binds more tightly to the DISC than full-length c-FLIP_{L}. c-FLIP_{S} is not processed by caspase 8 at the DISC. c-FLIP_{L} appears to be a more potent inhibitor of Fas-mediated cell death than c-FLIP_{S} (Irmler et al., 1997; Tschopp et al., 1998). Initially both pro-apoptotic and anti-apoptotic effects were proposed for c-FLIP. However, enhanced cell death occurred mainly in experiments using transient over-expression and may have been due to excessive levels of these DED-containing proteins, which may have caused clustering of other DED-containing proteins including procaspase 8, resulting in caspase activation (Siegel et al., 1998). The data from cell lines stably over-expressing c-FLIP and from mice deficient in c-FLIP support an anti-apoptotic function for c-FLIP (Yeh et al., 2000).

We found that c-FLIP_{L} was up-regulated and processed to its p43-form in MCF-7 cells following treatment with 5-FU and TDX. Furthermore, activation of caspase 8 and apoptosis in cells co-treated with chemotherapy and CH-11 coincided with processing of c-FLIP_{L}. These results suggested that c-FLIP_{L} regulated the onset of drug-induced Fas-mediated apoptosis in these cell lines. This hypothesis was further supported by data from overexpression and siRNA studies. c-FLIP overexpression abrogated the synergistic interaction between CH-11 and 5-FU, TDX, MTA and OXA by inhibiting caspase 8 activation. Furthermore, siRNA-targeting of both c-FLIP splice variants sensitised cells to these chemotherapeutic agents as determined by cell viability and PARP cleavage assays. Collectively, these results indicate that c-FLIP inhibits apoptosis in response to these drugs.

Surprisingly, we also found that siRNA-mediated down-regulation of c-FLIP_{L} and c-FLIP_{S} induced caspase 8 activation and PARP cleavage in the absence of co-treatment with chemotherapy (although co-treatment with drug enhanced the effect). The inventors found that overexpression of c-FLIP_{L} protected HCT116 cells from chemotherapy-induced apoptosis and apoptosis induced following co-treatment with chemotherapy and the Fas agonistic antibody CH-11. In addition to blocking caspase 8 activation, DISC-bound c-FLIP has been reported to promote activation of the ERK, PI3-kinase/Akt and NFκB signalling pathways (Kataoka et al., 2000; Panka et al., 2001). The NFκB, PI3K/Akt and ERK signal transduction pathways are associated with cell survival and/or proliferation, therefore, c-FLIP is capable of both blocking caspase 8 activation and also recruiting adaptor proteins that can activate intrinsic survival and proliferation pathways (Shu et al., 1997). Furthermore, c-FLIP also inhibits procaspase 8 activation at the DISCs formed by the TRAIL receptors DR4 and DR5 (Krueger et al., 2001). rTRAIL induces apoptosis in a range of human cancer cell lines including colorectal and breast, indicating that the TRAIL receptors are widely expressed in tumour cells (Ashkenazi, 2002). It is possible that expression of DR4 and DR5 is tolerated in tumours because c-FLIP converts the apoptotic signal to one which promotes survival and proliferation. Thus, siRNA-mediated down-regulation of c-FLIP may induce apoptosis by inhibiting FLIP-mediated activation of NFκB, PI3K/Akt and ERK and promoting activation of caspase 8 at TRAIL DISCS.

We have found that c-FLIP is a key regulator of Fas-mediated apoptosis in response to 5-FU, TS-targeted antifolates and OXA. Our results suggest that c-FLIP may be a clinically useful predictive marker of response to these agents and that c-FLIP is a therapeutically attractive target.

Furthermore, Our findings indicate that c-FLIP_{L} overexpression inhibits apoptosis of colorectal cancer cells in response to the chemotherapeutic agents used in the treatment of colorectal cancer (5-FU, oxaliplatin and CPT-11). This has particular clinical relevance given the high incidence of c-FLIP_{L} overexpression observed in colorectal cancer (6) and suggests that c-FLIP_{L} overexpression may contribute to chemoresistance in colorectal cancer. Interestingly, c-FLIP_{S} overexpression failed to protect colorectal cancer cells from chemotherapy-induced apoptosis, or apoptosis induced by co-treatment with chemotherapy and CH-11. These results would suggest that, of the two splice forms, c-FLIP_{L} is the more important mediator of resistance to chemotherapy in colorectal cancer cells.

Our study indicates that down-regulating c-FLIP in a panel of colorectal cancer cells that have not been selected for drug resistance increases their sensitivity to a range of cytotoxic drugs with differing mechanisms of action. Furthermore, the study has demonstrated that the down-regulation of c-FLIP alone can induce apoptosis.

It would appear from our c-FLIP overexpressing cell lines and studies using a c-FLIP_{L}-specific siRNA that the long splice form may be the more important in mediating survival of colorectal cancer cells, however conclusive proof of this will require the generation of a c-FLIP_{S}-specific siRNA. The induction of apoptosis following c-FLIP knock-down is most likely mediated by death receptors such as Fas and DR5. We have previously shown that Fas is up-regulated in response to 5-FU in HCT116p53^{+/+} and RKO cells, but not in HCT116p53^{-/-} and H630 cells (39), while DR5 is constitutively expressed in both HCT116 cell lines and the RKO and H630 lines (unpublished observations). It is possible that knocking down c-FLIP expression (either in the presence or absence of chemotherapy) removes c-FLIP-mediated inhibition of caspase 8 activation at Fas and/or DR5 DISCs, leading to caspase 8-mediated activation of apoptosis. Indeed, our initial evidence suggests that the onset of apoptosis and caspase 8 activation following c-FLIP knock-down are tightly coupled. In addition to blocking caspase 8 activation, DISC-bound c-FLIP has been reported to promote activation of the anti-apoptotic ERK, PI3-kinase/Akt and NF-κB signalling pathways (7, 8). So, it is also possible that loss of c-FLIP eliminates DISC-dependent up-regulation of these survival pathways, leading to enhanced susceptibility to apoptosis. In addition, a recent study has suggested that c-FLIP_{L} may have a non-DISC-dependent anti-apoptotic function by binding to and inhibiting pro-apoptotic signalling via p38 MAPK (40).

The p53 tumour suppressor gene is mutated in 40-60% of colorectal cancers most often in the central DNA-binding core domain responsible for sequence-specific binding to transcriptional target genes (41). p53 has been reported to both transcriptionally up-regulate c-FLIP (42) and target it for ubiquitin-mediated degradation by the proteasome (43), suggesting that the effect of p53 on c-FLIP expression is complex. In the present study, we consistently found that expression of both c-FLIP splice forms was higher in the p53 null HCT116 cell line compared to the isogenic p53 wild type line. We also examined how p53 status affected cell viability when c-FLIP was down-regulated. Although siRNA targeting of c-FLIP significantly enhanced chemotherapy-induced apoptosis in p53 null HCT116 cells, the effect was not as dramatic as in the p53 wild type line. Similarly, the induction of apoptosis after a 48 hour exposure to FLIP-targeted siRNA alone was greater in the p53 wild type setting. However, longer exposure times (72 hours) and higher concentrations (10-100 nM) of FT siRNA induced levels of apoptosis in the HCT116 p53 null cell line that approached those observed in the p53 wild type parental cell line. It is possible that the differential sensitivity of the p53 wild type and null cells to FT siRNA was at least partly due to the higher constitutive levels of c-FLIP expression in the p53 null line. It may also reflect lower levels of basal and chemotherapy-induced expression of the p53-regulated genes encoding the Fas and DR5 death receptors in the p53 null cell line, which lowers its sensitivity to loss of c-FLIP expression. Of note, down-regulation of c-FLIP markedly enhanced apoptosis in response to oxaliplatin in the p53 null cells, which are usually highly resistant to oxaliplatin (15). Further analyses revealed that the effects of targeting c-FLIP on chemotherapy-induced apoptosis were not confined to the HCT116 lines, as similar results were obtained in the p53 wild type RKO and p53 mutant H630 lines. Moreover, more potent knock down of c-FLIP with higher concentrations of siRNA triggered apoptosis in the absence of chemotherapy in both RKO and H630 cell lines. Collectively these results suggest that c-FLIP is an important regulator of cell survival in p53 wild type, null and mutant colorectal cancer cells in the presence and absence of chemotherapy.

These findings have direct clinical relevance as 5-FU/leucovorin/oxaliplatin (FOLFOX) and 5-FU/leucovorin/CPT-11 (FOLFIRI) combination chemotherapies are currently widely used in the treatment of advanced colorectal cancer, and FOLFOX has recently been demonstrated to improve 3-year survival compared to 5-FU/leucovorin in the adjuvant setting of the disease (78.2% versus 72.9%, p=0.002) (44). Furthermore, clinical studies have demonstrated significantly elevated c-FLIP expression in colorectal and gastric tumours (6, 45), suggesting that c-FLIP may not only be a relevant clinical target in colorectal cancer, but also in gastric cancer, where 5-FU-based chemotherapy regimens are also used. In conclusion, this study suggests that c-FLIP may represent an important clinical marker of drug resistance in colorectal cancer and that targeting c-FLIP, either alone, or in combination with standard chemotherapies has therapeutic potential for the treatment of this disease.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

Aherne, G. W., Hardcastle, A., Raynaud, F., and Jackman, A. L. (1996). Immunoreactive dUMP and TTP pools as an index of thymidylate synthase inhibition; effect of tomudex (ZD1694) and a nonpolyglutamated quinazoline antifolate (CB30900) in L1210 mouse leukaemia cells. Biochem Pharmacol 51, 1293-1301.
Ashkenazi, A. (2002). Targeting death and decoy receptors of the tumour-necrosis factor superfamily. Nat Rev Cancer 2, 420-430.
Bennett, M. W., O'Connell, J., O'Sullivan, G. C., Brady, C., Roche, D., Collins, J. K., and Shanahan, F. (1998). The Fas counterattack in vivo: apoptotic depletion of tumor-infiltrating lymphocytes associated with Fas ligand expression by human esophageal carcinoma. J Immunol 160, 5669-5675.
Giacchetti, S., Perpoint, B., Zidani, R., Le Bail, N., Faggiuolo, R., Focan, C., Chollet, P., Llory, J. F., Letourneau, Y., Coudert, B., et al. (2000). Phase III multicenter randomized trial of oxaliplatin added to chronomodulated fluorouracil-leucovorin as first-line treatment of metastatic colorectal cancer. J Clin Oncol 18, 136-147.
Hughes, L. R., Stephens, T. C., Boyle, F. T., and Jackman, A. L. (1999). Raltitrexed (Tomudex™), a highly polyglutamatable antifolate thymidylate synthase inhibitor, Humana press).
Irmler, M., Thome, M., Hahne, M., Schneider, P., Hofmann, K., Steiner, V., Bodmer, J. L., Schroter, M., Burns, K., Mattmann, C., et al. (1997). Inhibition of death receptor signals by cellular FLIP. Nature 388, 190-195.
Kataoka, T., Budd, R. C., Holler, N., Thome, M., Martinon, F., Irmler, M., Burns, K., Hahne, M., Kennedy, N., Kovacsovics, M., and Tschopp, J. (2000). The caspase-8 inhibitor FLIP promotes activation of NF-kappaB and Erk signaling pathways. Curr Biol 10, 640-648.
Kern, D. H., Morgan, C. R., and Hildebrand-Zanki, S. U. (1988). In vitro pharmacodynamics of 1-beta-D-arabinofuranosylcytosine: synergy of antitumor activity with cis-diamminedichloroplatinum(II). Cancer Res 48, 117-121.
Krueger, A., Baumann, S., Krammer, P. H., and Kirchhoff, S. (2001). FLICE-inhibitory proteins: regulators of death receptor-mediated apoptosis. Mol Cell Biol 21, 8247-8254.
Longley, D. B., Boyer, J., Allen, W. L., Latif, T., Ferguson, P. R., Maxwell, P. J., McDermott, U., Lynch, M., Harkin, D. P., and Johnston, P. G. (2002). The role of thymidylate synthase induction in modulating p53-regulated gene expression in response to 5-fluorouracil and antifolates. Cancer Res 62, 2644-2649.
Longley, D. B., Ferguson, P. R., Boyer, J., Latif, T., Lynch, M., Maxwell, P., Harkin, D. P., and Johnston, P. G. (2001). Characterization of a thymidylate synthase (TS)-inducible cell line: a model system for studying sensitivity to TS- and non-TS-targeted chemotherapies. Clin Cancer Res 7, 3533-3539.
Longley, D. B., Harkin, D. P., and Johnston, P. G. (2003). 5-fluorouracil: mechanisms of action and clinical strategies. Nat Rev Cancer 3, 330-338.
Mosmann, T. (1983). Rapid calorimetric assay for cellular growth and survival: application to proliferation and cytotoxicity assays. J Immunol Methods 65, 55-63.
Nagata, S. (1999). Fas ligand-induced apoptosis. Annu Rev Genet. 33, 29-55.
Niikura, Y., Nonaka, T., and Imajoh-Ohmi, S. (2002). Monitoring of caspase-8/FLICE processing and activation upon Fas stimulation with novel antibodies directed against a cleavage site for caspase-8 and its substrate, FLICE-like inhibitory protein (FLIP). J Biochem (Tokyo) 132, 53-62.
O'Connell, J., Bennett, M. W., O'Sullivan, G. C., Collins, J. K., and Shanahan, F. (1997). The Fas counterattack: a molecular mechanism of tumor immune privilege. Mol Med 3, 294-300.
O'Connell, J., Bennett, M. W., O'Sullivan, G. C., Collins, J. K., and Shanahan, F. (1999). Resistance to Fas (APO-1/CD95)-mediated apoptosis and expression of Fas ligand in esophageal cancer: the Fas counterattack. Dis Esophagus 12, 83-89.
Panka, D. J., Mano, T., Suhara, T., Walsh, K., and Mier, J. W. (2001). Phosphatidylinositol 3-kinase/Akt activity regulates c-FLIP expression in tumor cells. J Biol Chem 276, 6893-6896.
Romanelli, S., Perego, P., Pratesi, G., Carenini, N., Tortoreto, M., and Zunino, F. (1998). In vitro and in vivo interaction between cisplatin and topotecan in ovarian carcinoma systems. Cancer Chemother Pharmacol 41, 385-390.
Ryu, B. K., Lee, M. G., Chi, S. G., Kim, Y. W., and Park, J. H. (2001). Increased expression of cFLIP(L) in colonic adenocarcinoma. J Pathol 194, 15-19.
Scaffidi, C., Schmitz, I., Krammer, P. H., and Peter, M. E. (1999). The role of c-FLIP in modulation of CD95-induced apoptosis. J Biol Chem 274, 1541-1548.
Shih, C., Chen, V. J., Gossett, L. S., Gates, S. B., MacKellar, W. C., Habeck, L. L., Shackelford, K. A., Mendelsohn, L. G., Soose, D. J., Patel, V. F., et al. (1997). LY231514, a pyrrolo[2,3-d]pyrimidine-based antifolate that inhibits multiple folate-requiring enzymes. Cancer Res 57, 1116-1123.
Shu, H. B., Halpin, D. R., and Goeddel, D. V. (1997). Casper is a FADD- and caspase-related inducer of apoptosis. Immunity 6, 751-763.
Siegel, R. M., Martin, D. A., Zheng, L., Ng, S. Y., Bertin, J., Cohen, J., and Lenardo, M. J. (1998). Death-effector filaments: novel cytoplasmic structures that recruit caspases and trigger apoptosis. J Cell Biol 141, 1243-1253.
Tschopp, J., Irmler, M., and Thome, M. (1998). Inhibition of fas death signals by FLIPs. Curr Opin Immunol 10, 552-558.
Yeh, W. C., Itie, A., Elia, A. J., Ng, M., Shu, H. B., Wakeham, A., Mirtsos, C., Suzuki, N., Bonnard, M., Goeddel, D. V., and Mak, T. W. (2000). Requirement for Casper (c-FLIP) in regulation of death receptor-induced apoptosis and embryonic development. Immunity 12, 633-642.
Aherne, G. W., Hardcastle, A., Raynaud, F., and Jackman, A. L. (1996). Immunoreactive dUMP and TTP pools as an index of thymidylate synthase inhibition; effect of tomudex (ZD1694) and a nonpolyglutamated quinazoline antifolate (CB30900) in L1210 mouse leukaemia cells. Biochem Pharmacol 51, 1293-1301.
Bennett, M., Macdonald, K., Chan, S. W., Luzio, J. P., Simari, R., and Weissberg, P. (1998a). Cell surface trafficking of Fas: a rapid mechanism of p53-mediated apoptosis. Science 282, 290-293.
Bennett, M. W., O'Connell, J., O'Sullivan, G. C., Brady, C., Roche, D., Collins, J. K., and Shanahan, F. (1998b). The Fas counterattack in vivo: apoptotic depletion of tumor-infiltrating lymphocytes associated with Fas ligand expression by human esophageal carcinoma. J Immunol 160, 5669-5675.
Bunz, F., Hwang, P. M., Torrance, C., Waldman, T., Zhang, Y., Dillehay, L., Williams, J., Lengauer, C., Kinzler, K. W., and Vogelstein, B. (1999). Disruption of p53 in human cancer cells alters the responses to therapeutic agents. J Clin Invest 104, 263-269.
Chou, T. C., and Talalay, P. (1984). Quantitative analysis of dose-effect relationships: the combined effects of multiple drugs or enzyme inhibitors. Adv Enzyme Regul 22, 27-55.
Chu, E., Koeller, D. M., Johnston, P. G., Zinn, S., and Allegra, C. J. (1993). Regulation of thymidylate synthase in human colon cancer cells treated with 5-fluorouracil and interferon-gamma. Mol Pharmacol 43, 527-533.
Giacchetti, S., Perpoint, B., Zidani, R., Le Bail, N., Faggiuolo, R., Focan, C., Chollet, P., Llory, J. F., Letourneau, Y., Coudert, B., et al. (2000). Phase III multicenter randomized trial of oxaliplatin added to chronomodulated fluorouracil-leucovorin as first-line treatment of metastatic colorectal cancer. J Clin Oncol 18, 136-147.
Hughes, L. R., Stephens, T. C., Boyle, F. T., and Jackman, A. L. (1999). Raltitrexed (Tomudex™), a highly polyglutamatable antifolate thymidylate synthase inhibitor, Humana press).
Ichikawa, K., Yoshida-Kato, H., Ohtsuki, M., Ohsumi, J., Yamaguchi, J., Takahashi, S., Tani, Y., Watanabe, M., Shiraishi, A., Nishioka, K., et al. (2000). A novel murine anti-human Fas mAb which mitigates lymphadenopathy without hepatotoxicity. Int Immunol 12, 555-562.
Jiang, S., Song, M. J., Shin, E. C., Lee, M. O., Kim, S. J., and Park, J. H. (1999). Apoptosis in human hepatoma cell lines by chemotherapeutic drugs via Fas-dependent and Fas-independent pathways. Hepatology 29, 101-110.
Kano, Y., Ohnuma, T., Okano, T., and Holland, J. F. (1988). Effects of vincristine in combination with methotrexate and other antitumor agents in human acute lymphoblastic leukemia cells in culture. Cancer Res 48, 351-356.
Kern, D. H., Morgan, C. R., and Hildebrand-Zanki, S. U. (1988). In vitro pharmacodynamics of 1-beta-D-arabinofuranosylcytosine: synergy of antitumor activity with cis-diamminedichloroplatinum(II). Cancer Res 48, 117-121.
Krueger, A., Baumann, S., Krammer, P. H., and Kirchhoff, S. (2001). FLICE-inhibitory proteins: regulators of death receptor-mediated apoptosis. Mol Cell Biol 21, 8247-8254.
Longley, D. B., Boyer, J., Allen, W. L., Latif, T., Ferguson, P. R., Maxwell, P. J., McDermott, U., Lynch, M., Harkin, D. P., and Johnston, P. G. (2002a). The role of thymidylate synthase induction in modulating p53-regulated gene expression in response to 5-fluorouracil and antifolates. Cancer Res 62, 2644-2649.
Longley, D. B., Ferguson, P. R., Boyer, J., Latif, T., Lynch, M., Maxwell, P., Harkin, D. P., and Johnston, P. G. (2001). Characterization of a thymidylate synthase (TS)-inducible cell line: a model system for studying sensitivity to TS- and non-TS-targeted chemotherapies. Clin Cancer Res 7, 3533-3539.
Longley, D. B., Harkin, D. P., and Johnston, P. G. (2003). 5-fluorouracil: mechanisms of action and clinical strategies. Nat Rev Cancer 3, 330-338.
Longley, D. B., McDermott, U., and Johnston, P. G. (2002b). Clinical significance of prognostic and predictive markers in colorectal cancer. Pharmacogenomics J 2, 209-216.
Mosmann, T. (1983). Rapid calorimetric assay for cellular growth and survival: application to proliferation and cytotoxicity assays. J. Immunol. Methods 65, 55-63.
Muller, M., Wilder, S., Bannasch, D., Israeli, D., Lehlbach, K., Li-Weber, M., Friedman, S. L., GaLle, P. R., Stremmel, W., Oren, M., and Krammer, P. H. (1998). p53 activates the CD95 (APO-1/Fas) gene in response to DNA damage by anticancer drugs. J Exp Med 188, 2033-2045.
Munsch, D., Watanabe-Fukunaga, R., Bourdon, J. C., Nagata, S., May, E., Yonish-Rouach, E., and Reisdorf, P. (2000). Human and mouse Fas (APO-1/CD95) death receptor genes each contain a p53-responsive element that is activated by p53 mutants unable to induce apoptosis. J Biol Chem 275, 3867-3872.
Nagata, S. (1999). Fas ligand-induced apoptosis. Annu Rev Genet. 33, 29-55. O'Connell, J., Bennett, M. W., O'Sullivan, G. C., Collins, J. K., and Shanahan, F. (1997). The Fas counterattack: a molecular mechanism of tumor immune privilege. Mol Med 3, 294-300.
O'Connell, J., Bennett, M. W., O'Sullivan, G. C., Collins, J. K., and Shanahan, F. (1999). Resistance to Fas (APO-1/CD95)-mediated apoptosis and expression of Fas ligand in esophageal cancer: the Fas counterattack. Dis Esophagus 12, 83-89.
Pitti, R. M., Marsters, S. A., Lawrence, D. A., Roy, M., Kischkel, F. C., Dowd, P., Huang, A., Donahue, C. J., Sherwood, S. W., Baldwin, D. T., et al. (1998). Genomic amplification of a decoy receptor for Fas ligand in lung and colon cancer. Nature 396, 699-703.
Romanelli, S., Perego, P., Pratesi, G., Carenini, N., Tortoreto, M., and Zunino, F. (1998). In vitro and in vivo interaction between cisplatin and topotecan in ovarian carcinoma systems. Cancer Chemother Pharmacol 41, 385-390.
Ruberti, G., Cascino, I., Papoff, G., and Eramo, A. (1996). Fas splicing variants and their effect on apoptosis. Adv Exp Med Biol 406, 125-134.
Ryu, B. K., Lee, M. G., Chi, S. G., Kim, Y. W., and Park, J. H. (2001). Increased expression of cFLIP(L) in colonic adenocarcinoma. J Pathol 194, 15-19.
Sato, T., Irie, S., Kitada, S., and Reed, J. C. (1995). FAP-1: a protein tyrosine phosphatase that associates with Fas. Science 268, 411-415.
Shih, C., Chen, V. J., Gossett, L. S., Gates, S. B., MacKellar, W. C., Habeck, L. L., Shackelford, K. A., Mendelsohn, L. G., Soose, D. J., Patel, V. F., et al. (1997). LY231514, a pyrrolo[2,3-d]pyrimidine-based antifolate that inhibits multiple folate-requiring enzymes. Cancer Res 57, 1116-1123.
Swain, S. M., Lippman, M. E., Egan, E. F., Drake, J. C., Steinberg, S. M., and Allegra, C. J. (1989). Fluorouracil and high-dose leucovorin in previously treated patients with metastatic breast cancer. J Clin Oncol 7, 890-899.
Tillman, D. M., Petak, I., and Houghton, J. A. (1999). A Fas-dependent component in 5-fluorouracil/leucovorin-induced cytotoxicity in colon carcinoma cells. Clin Cancer Res 5, 425-430.
1. Nagata, S. Fas ligand-induced apoptosis. Annu Rev Genet, 33: 29-55, 1999.
2. Griffith, T. S, and Lynch, D. H. TRAIL: a molecule with multiple receptors and control mechanisms. Curr Opin Immunol, 10: 559-563, 1998.
6. Ryu, B. K., Lee, M. G., Chi, S. G., Kim, Y. W., and Park, J. H. Increased expression of cFLIP(L) in colonic adenocarcinoma. J Pathol, 194: 15-19, 2001.
7. Park, S. J., Kim, Y. Y., Ju, J. W., Han, B. G., Park, S. I., and Park, B. J. Alternative splicing variants of c-FLIP transduce the differential signal through the Raf or TRAF2 in TNF-induced cell proliferation. Biochem Biophys Res Commun, 289: 1205-1210, 2001.
14. Chou, T. C. and Talalay, P. Quantitative analysis of dose-effect relationships: the combined effects of multiple drugs or enzyme inhibitors. Adv Enzyme Regul, 22: 27-55, 1984.
15. Boyer, J., McLean, E. G., Aroori, S., Wilson, P., McCulla, A., Carey, P. D., Longley, D. B., and Johnston, P. G. Characterization of p53 wild-type and null isogenic colorectal cancer cell lines resistant to 5-fluorouracil, oxaliplatin and irinotecan. Clin Cancer Res, 10, 2004.
16. Bannerman, D. D., Tupper, J. C., Ricketts, W. A., Bennett, C. F., Winn, R. K., and Harlan, J. M. A constitutive cytoprotective pathway protects endothelial cells from lipopolysaccharide-induced apoptosis. J Biol Chem, 276: 14924-14932, 2001.
39. Longley, D. B., Allen, W. L., McDermott, U., Wilson, T. R., Latif, T., Boyer, J., Lynch, M., and Johnston, P. G. The roles of thymidylate synthase and p53 in regulating Fas-mediated apoptosis in response to antimetabolites. Clin Cancer Res, 10: 3562-3571, 2004.
40. Grambihler, A., Higuchi, H., Bronk, S. F., and Gores, G. J. cFLIP-L inhibits p38 MAPK activation: an additional anti-apoptotic mechanism in bile acid-mediated apoptosis. J Biol Chem, 278: 26831-26837, 2003.
41. Levine, A. J., Momand, J., and Finlay, C. A. The p53 tumour suppressor gene. Nature, 351: 453-456, 1991.
42. Bartke, T., Siegmund, D., Peters, N., Reichwein, M., Henkler, F., Scheurich, P., and Wajant, H. p53 upregulates cFLIP, inhibits transcription of NF-kappaB-regulated genes and induces caspase-8-independent cell death in DLD-1 cells. Oncogene, 20: 571-580, 2001.
43. Fukazawa, T., Fujiwara, T., Uno, F., Teraishi, F., Kadowaki, Y., Itoshima, T., Takata, Y., Kagawa, S., Roth, J. A., Tschopp, J., and Tanaka, N. Accelerated degradation of cellular FLIP protein through the ubiquitin-proteasome pathway in p53-mediated apoptosis of human cancer cells. Oncogene, 20: 5225-5231, 2001.
44. Andre, T., Boni, C., Mounedji-Boudiaf, L., Navarro, M., Tabernero, J., Hickish, T., Topham, C., Zaninelli, M., Clingan, P., Bridgewater, C., Tabah-Fisch, I., and de Gramont, A. Oxaliplatin, fluorouracil, and leucovorin as adjuvant treatment for colon cancer. N Engl J. Med. 350: 2343-2351, 2004.
45. Zhou, X. D., Yu, J. P., Liu, J., Luo, H. S., Chen, H. X., and Yu, H. G. Overexpression of cellular FLICE-inhibitory protein (FLIP) in gastric adenocarcinoma. Clin Sci (Lond), 106: 397-405, 2004.

1. A method of killing cancer cells, comprising administration to said cells of an effective amount of a c-FLIP inhibitor, wherein the c-FLIP inhibitor is administered as the sole cytotoxic agent in the substantial absence of other cytotoxic agents.

2. A method of treating cancer comprising administration to a subject in need thereof a therapeutically effective amount of a c-FLIP inhibitor, wherein the c-FLIP inhibitor is administered as the sole cytotoxic agent in the substantial absence of other cytotoxic agents.

3. A method of killing cancer cells having a p53 mutation, comprising administration to said cells of:
(a) a c-FLIP inhibitor and
(b) a chemotherapeutic agent, wherein the chemotherapeutic agent is a thymidylate synthase inhibitor, a platinum cytotoxic agent or a topoisomerase inhibitor.

4. A method of treating cancer associated with a p53 mutation comprising administration to a subject in need thereof
(a) a c-FLIP inhibitor and
(b) a chemotherapeutic agent, wherein the chemotherapeutic agent is a thymidylate synthase inhibitor, a platinum cytotoxic agent or a topoisomerase inhibitor.

5. The method according to claim 3, further comprising administration of:
(c) a death receptor binding member.

6. The method according to claim 5, wherein the death receptor is FAS.

7. The method according to claim 6, wherein the binding member is the FAS antibody CH11.

8. The method according to claim 3, wherein the chemotherapeutic agent is 5-FU, oxaliplatin or CPT-11.

9. The method according to claim 8, wherein the chemotherapeutic agent is 5-FU or oxaliplatin.

10. The method according to claim 4, wherein the c-FLIP inhibitor and the chemotherapeutic agent are administered in a potentiating ratio.

11. The method according to claim 10, wherein the c-FLIP inhibitor and the chemotherapeutic agent are administered in concentrations sufficient to produce a CI of less than 0.85.

12. The method according to claim 4, wherein the p53 mutation is such that p53 is completely inactivated in the cancer cells.

13. The method according to claim 4, wherein the p53 mutation is a missense mutation resulting in the substitution of histidine (R175H mutation) or a missense mutation resulting in the substitution of tryptophan (R248W mutation) for arginine.

14. The method according to claim 2, wherein said c-FLIP inhibitor is an RNAi agent, which modulates expression of a c-FLIP gene.

15. The method according to claim 14 wherein the c-FLIP inhibitor is an RNAi agent having nucleotide sequence

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A pharmaceutical composition for the treatment of cancer, wherein the composition comprises a c-FLIP inhibitor as the sole cytotoxic agent and a pharmaceutically acceptable excipient, diluent or carrier, wherein the composition is for treatment in the absence of other cytotoxic agents.

30. A pharmaceutical composition for the treatment of a cancer associated with a p53 mutation, wherein the composition comprises
(a) a c-FLIP inhibitor
(b) a chemotherapeutic agent, wherein the chemotherapeutic agent is a thymidylate synthase inhibitor, a platinum cytotoxic agent or a topoisomerase I inhibitor and
(c) a pharmaceutically acceptable excipient, diluent or carrier.

31. The composition according to claim 30, further comprising a death receptor binding member.

32. The composition according to claim 31, wherein the death receptor is FAS.

33. The composition according to claim 32, wherein the binding member is the FAS antibody CH11.

34. The composition according to claim 30, wherein the chemotherapeutic agent is 5-FU, oxaliplatin or CPT-11.

35. The composition according to claim 34, wherein the chemotherapeutic agent is 5-FU or oxaliplatin.

36. The composition according to claim 30, wherein the c-FLIP inhibitor and the chemotherapeutic agent are present in a potentiating ratio.

37. The composition according to claim 36, wherein the c-FLIP inhibitor and the chemotherapeutic agent are present in concentrations sufficient to produce a CI of less than 0.85.

38. The composition according to claim 30, wherein the p53 mutation is such that p53 is completely inactivated in the cancer cells.

39. The composition according to claim 30, wherein the p53 mutation is a missense mutation resulting in the substitution of histidine (R175H mutation) or a missense mutation resulting in the substitution of tryptophan (R248W mutation) for arginine.

40. The composition according to claim 29, wherein said c-FLIP inhibitor is an RNAi agent, which modulates expression of a c-FLIP gene.

41. The composition according to claim 40 wherein the c-FLIP inhibitor is an RNAi agent having nucleotide sequence

42. A kit for the treatment of cancer associated with a p53 mutation, said kit comprising
(a) a c-FLIP inhibitor and
(b) a chemotherapeutic agent, wherein the chemotherapeutic agent is a thymidylate synthase inhibitor, a platinum cytotoxic agent or a topoisomerase I inhibitor and
(c) instructions for the administration of (a) and (b) separately, sequentially or simultaneously.

43. An RNAi agent having nucleotide sequence

| | |
|---|---|
| AAG CAG TCT GTT CAA GGA GCA | (SEQ ID NO: 1) |
| or | |
| AAG GAA CAG CTT GGC GCT CAA. | (SEQ ID NO: 2) |

44. An RNAi agent consisting of nucleotide sequence

| | |
|---|---|
| AAG CAG TCT GTT CAA GGA GCA | (SEQ ID NO: 1) |
| or | |
| AAG GAA CAG CTT GGC GCT CAA. | (SEQ ID NO: 2) |

45. The method according to claim 4, wherein said c-FLIP inhibitor is an RNAi agent, which modulates expression of a c-FLIP gene.

46. The method according to claim 45, wherein the c-FLIP inhibitor is an RNAi agent having nucleotide sequence

| | |
|---|---|
| AAG CAG TCT GTT CAA GGA GCA | (SEQ ID NO: 1) |
| or | |
| AAG GAA CAG CTT GGC GCT CAA. | (SEQ ID NO: 2) |

47. The composition according to claim 30, wherein said c-FLIP inhibitor is an RNAi agent, which modulates expression of a c-FLIP gene.

48. The composition according to claim 47, wherein the c-FLIP inhibitor is an RNAi agent having nucleotide sequence

| | |
|---|---|
| AAG CAG TCT GTT CAA GGA GCA | (SEQ ID NO: 1) |
| or | |
| AAG GAA CAG CTT GGC GCT CAA. | (SEQ ID NO: 2) |
